(19) United States
(12) Patent Application Publication
(10) Pub. No.: US 2024/0189577 A1
(43) Pub. Date: Jun. 13, 2024

(54) METHODS AND DEVICES FOR DIRECTING DISSIPATION OF A CHARGED ACTIVE AGENT AFTER ADMINISTRATION

(71) Applicant: REGRESAR MEDICAL, INC., San Diego, CA (US)

(72) Inventors: Hal W. Gibson, Long Beach, CA (US); Raymond Wagenknecht, San Diego, CA (US)

(21) Appl. No.: 18/406,517

(22) Filed: Jan. 8, 2024

Related U.S. Application Data
(63) Continuation-in-part of application No. 17/206,942, filed on Mar. 19, 2021.
Application No. 17/206,942 is a continuation of application No. 16/890,580, filed on Jun. 2, 2020, now Pat. No. 11,129,979.
Application No. 16/890,580 is a continuation of application No. PCT/US2020/018979, filed on Feb. 20, 2020.
(63) Continuation-in-part of application No. PCT/US2022/036147, filed on Jul. 5, 2022.
(60) Provisional application No. 62/807,929, filed on Feb. 20, 2019.
(60) Provisional application No. 62/812,228, filed on Feb. 28, 2019.
(60) Provisional application No. 62/878,030, filed on Jul. 24, 2019.
(60) Provisional application No. 63/220,214, filed on Jul. 9, 2021.

(51) Int. Cl. A61N 1/04; A61K 31/00; A61K 31/185; A61M 35/00; A61N 2/00; G01N 33/50

(52) U.S. Cl. CPC A61N 1/0456; A61M 35/00; A61N 1/0452; A61N 2/002; A61K 31/00; A61K 31/185; G01N 33/50

(57) ABSTRACT

Devices and their use for directing dissipation of a charged active agent after injection, the device having two electrodes spaced apart from one another and configured to be applied to the skin surface so that a directional electric field can be generated between the two electrodes within the dermal and/or subcutaneous layer, the flexible substrate having either a through gap sized for permitting passage of an injection device and optionally positioned between the two electrodes; or includes a tearaway portion forming a through gap for permitting passage of an injection device and optionally between the two electrodes.

### CROSS REFERENCE TO RELATED APPLICATIONS

This is a continuation in part of U.S. patent application Ser. No. 17/206,942 filed Mar. 19, 2021, which is a continuation of U.S. patent application Ser. No. 16/990,580 filed Jun. 2, 202, now U.S. Pat. No. 11,129,979, which is a continuation of international application no. PCT/US2020/018979 filed Feb. 20, 2020, which claims benefit of priority to U.S. provisional patent application No. 62/807,929, filed Feb. 20, 2019 and U.S. provisional patent application No. 62/812,228 filed Feb. 28, 2019 and U.S. provisional patent application No. 62/878,030 filed Jul. 24, 2019; the content of each is incorporated herein by reference in its entirety.

This is also a continuation in part of international application no. PCT/2022/036147 filed Jul. 5, 2022, which claims priority to U.S. provisional patent application No. 63/220,214 filed Jul. 9, 2021, each of which is incorporated by reference herein in its entirety.

### TECHNICAL FIELD

The invention relates generally to the field of medical devices and more specifically to a device for directing the dissipation of a charged active agent by way of an applied electric field after administration.

### BACKGROUND

Materials can be administered to the body in a number of ways. Among these, injection is one of the more common methods of introducing therapeutic materials into patients. However, injections carry the risk of unwanted dispersion or dissipation of the materials beyond the desired treatment site. This is exacerbated in instances of poor injection technique and when understanding of the anatomy at the injection site is limited. For example, after administering neurotoxins (e.g. BOTOX) for cosmetic treatments to remove glabellar lines or crow's feet, dissipation of the neurotoxin towards the eye can cause eye droop or “ptosis”. Ptosis complications typically become apparent within 2-10 days of treatment and can last up to 4 to 6 weeks. Eyebrow ptosis is one of the more common complications in these cases; however, eyelid ptosis can also occur if the toxin dissipates into the levator palpebrae. This can cause not only the unwanted aesthetic of a “droopy eye” but also a decrease in the field of vision, which can lead to injury. Also, unwanted dissipation can affect the tear glands. Neck weakness or difficulty swallowing (dysphagia) can occur as a result of injections to the jaw. Similarly, large doses of neurotoxin injected into the platysma for the treatment of platysmal bands and horizontal neck lines can produce weakness of the neck flexors and dysphagia and hoarseness. Further, immune response can be increased when injected materials spread from desired treatment sites. In the case of dermal filler injections, the injected material can spread beyond the treatment site, causing unwanted cosmetic effects, for example when treating the lip or cheeks.

Iontophoresis provides an alternative to injection. It delivers molecules across the skin barrier using a repulsive electric force. However, traditional methods of iontophoresis do not offer the precision of injection and thus can also suffer from more complications than injection.

Accordingly, there remains a need to provide new approaches that limit the unwanted dissipation of active agents away from their desired treatment area.

### SUMMARY

The inventions described herein address the above deficiencies and provide related benefits. In particular, this disclosure describes devices that direct dissipation of active agents away from regions that are likely to cause complications and towards regions that improve the intended therapeutic treatment.

This is accomplished through devices, systems and methods that apply electromagnetic forces such as magnetism, electric charge, electric field, and/or electric current to the skin. By directing such forces approximately parallel to the skin and between opposing electrodes, the present disclosure demonstrates that charged compositions or active agents can be selectively moved towards regions where exposure to the composition or agent is desired and away from regions where exposure is not desired. An exemplary device includes two electrodes spaced apart from one another on a flexible substrate configured for placement on the skin so that a directional electric field can be generated between the two electrodes and thus generally parallel to the skin. The flexible substrate is configured for placement against the skin of the subject and includes either a through gap sized for permitting passage of an injection device, which is most often between the two electrodes but in some instances can be at the attractive electrode; or includes a tearaway portion configured to tearaway to form a through gap sized for permitting passage of an injection device, which is also most often between the electrodes but in some instances can be at the attractive electrode. Exemplary injection devices include syringe/needle and iontophoresis units.

In embodiments where injection is to occur at the forehead, such as during cosmetic procedures or when used with migraine therapies, the flexible substrate can be shaped for placement above the eyes of the subject so that the through gap is positioned to permit intramuscular injection of a charged composition at the subject's forehead.

In embodiments including the tearaway portion for forming a through gap, such as between the two electrodes, the substrate may have a tearaway perforation that extends between the two electrodes. In such embodiments, the perforation can be torn to form the gap while the two electrodes remain in place or the perforation can be torn so that the two electrodes can be pulled away from one another so that the gap enlarges as the two electrodes are pulled away from each other.

In embodiments that include or do not include the tearaway perforation, the flexible substrate can include a tether that tethers the electrodes together.

In some embodiments, a first of the two electrodes is encircled by a second of the two electrodes. In other embodiments, the two electrodes are substantially parallel to one another. In other embodiments the two electrodes are positioned to follow different portions of the subject, such as one above the eyebrow and one below the hairline.

In some embodiments, the device also includes a power supply that supplies opposing charges to the two electrodes to power a directional electric field. In other embodiments, the power supply is provided in a separate system, where the system includes the device(s) as set forth in this document.

In some embodiments, the power supply plugs into a power outlet or is a remote power supply. In other embodiments the power supply is battery powered and attached to a same flexible substrate as at least one of the two electrodes. In some embodiments, the power supply is configured for mounting to the subject's face or head, such as via a headband, mask or through use of an adhesive.

In some embodiments, the device includes electrode connectors that differ in shape between the two electrodes. For example, a negative electrode connector can differ from a positive electrode connector such that the negative electrode can only be connected to the negative “lead” and the positive electrode can only be connected to the positive lead.

In a related aspect of the invention, a device for directing dissipation of a charged composition after injection is provided, the device including two electrodes spaced apart from one another on a perforated substrate that is configured to tear away to separate the two electrodes from one another, thereby forming a through gap sized to permit passage of an injection device, the substrate having an adhesive backing configured for positioning the two electrodes against the skin of a subject for the generation of a directional electric field approximately parallel to the skin.

In another related aspect of the invention, use of the device for the treatment of facial wrinkles is provided. In some embodiments, the device is applied above the eyes of the subject, the subject receives an injection of a charged active agent through the through gap, and opposing charges are applied to the two electrodes to create the directional electric field approximately parallel to the skin and so that the polarity encourages dissipation of the active agent away from the eyes of the subject.

In some embodiments, the charged active agent includes a neurotoxin, optionally a botulinum toxin (BoNT) selected from the group consisting of BoNT/A, BoNT/B, BoNT/C, BoNT/D, BONT/E, BONT/F, and BoNT/G. The botulinum toxin can include, for example, the 900 kd BoNT/A complex, the 150 kd neurotoxin component dissociated from the 900 kd BoNT/A complex, or combinations thereof.

In some embodiments, the opposing charges are pulsed.

In another related aspect of the invention use of the device for the treatment of migraine is provided. In such embodiments, the device can be positioned at a muscle selected from the group consisting of the procerus muscle, the corrugator muscle, the frontalis muscle, the occipitalis muscle, and the trapezius muscle; and the subject receives an injection of a charged active agent for the treatment of the migraine through the through gap. Exemplary active agents for use in migraine treatment include botulinum toxin (BoNT), such as but not limited to BoNT/A, BoNT/E, and BoNT/B.

In still another related aspect of the invention, a method of directing dissipation of a charged active agent in a subject is provided, which includes applying the a device as disclosed herein against the skin of the subject, injecting a charged active agent through the through gap, and applying opposing charges to the two electrodes, thereby creating a directional electric field between the two electrodes and approximately parallel to the skin so that the polarity encourages dissipation of the active agent towards one of the two electrodes and away from another.

In yet another related aspect of the invention, a method for directing dissipation of a charged active agent is provided, which includes placing two electrodes against a subject's skin, the two electrodes configured to receive opposing charges to generate a directional electric field between the two electrodes and approximately parallel to the skin; subcutaneously or intramuscularly administering the charged active agent between the two electrodes, the charged active agent having a net negative or net positive charge at physiological pH; and applying a positive charge to one of the two electrodes and a negative charge to another of the two electrodes to produce the directional electric field, thereby electrically directing dissipation of the administered charged active agent towards one of the two electrodes and away from another of the two electrodes approximately parallel to the skin.

In still another related aspect of the invention, a method for directing dissipation of a charged active agent is provided, the method including: subcutaneously or intramuscularly administering a charged active agent into a subject, the charged active agent having a net negative or net positive charge at physiological pH; placing two electrodes against the subject's skin, the two electrodes configured to receive opposing charges to generate a directional electric field approximately parallel to the skin and at the administered charged active agent; and applying a positive charge to one of the two electrodes and a negative charge to another of the two electrodes to produce the directional electric field, thereby electrically directing dissipation of the administered charged active agent towards one of the two electrodes and away from the oppositely charged electrode.

### BRIEF DESCRIPTION OF THE DRAWINGS

FIGS. 1A-1B are top and side views respectively of a non-limiting exemplary device 10 for directing dissipation of a charged active agent after injection including a substrate 12, an attractive electrode 20, a repellent electrode 30 and a through gap 40 embodied as a through bore 41 for accessing a treatment site after applying the device 10 to the skin. The side view in FIG. 1B more clearly shows directional forces encouraging dissipation parallel to the skin 5 once applied, which is towards the attractive electrode 20 and away from the repellent electrode 30.

FIGS. 1C-1D are top and side views respectively of another non-limiting exemplary device 10 for directing dissipation of a charged active agent after injection including a substrate 12, an attractive electrode 20, a repellent electrode 30 and a through gap 40 embodied as through slits 42 for accessing an injection site after applying the device 10 to the skin. The side view in FIG. 1D more clearly shows directional forces encouraging dissipation parallel to the skin 5 and towards the attractive electrode 20 and away from the repellent electrode 30.

FIG. 1E is a top view of another non-limiting exemplary device 10 for directing dissipation of a charged active agent after injection including a substrate 12, an attractive electrode 20, a repellent electrode 30 and a through gap 40 for accessing a treatment site after applying the device 10 to the skin

FIGS. 2A-2F depict additional non-limiting exemplary devices 10 for directing dissipation of a charged active agent after injection including a substrate 12, an attractive electrode 20, and a repellent electrode 30 for applying electric charge through the skin. A flexible tether 44 ensures the corresponding electrodes 20, 30 remain properly grouped.

FIGS. 3A-3B depict additional non-limiting exemplary devices 10 for directing dissipation of a charged active agent after injection for the treatment of glabellar lines, including a substrate 12 with perforation 16, an attractive electrode 20, a repellent electrode 30. FIG. 3C depicts an additional non-limiting exemplary device 10 for directing dissipation of a charged active agent after injection including an adhesive backed substrate 12 shared by an attractive electrode 20, a repellent electrode 30, and a battery 50 with perforations 16 that tear away to separate the attractive electrode 20, repellent electrode 30, and battery 50 so that they remain tethered only by leads 25.

FIG. 4 depicts a non-limiting system 100 for directing dissipation of a charged active agent after injection including an attractive electrode 20, a repellent electrode 30 and a power supply 72 configured as an EMS unit 70.

FIG. 5 depicts a non-limiting exemplary electrode configuration for use when directing dissipation of a charged active agent after injection for the treatment of both frown or glabellar lines and crow's feet.

FIG. 6 depicts a non-limiting exemplary electrode configuration for use when directing dissipation of a charged active agent after injection for the treatment both of forehead lines and crow's feet.

FIG. 7 depicts attractive and repellent electrodes applied to a subject's face prior to administration of botulinum treatment.

FIG. 8 depicts a starch test showing the results of a botulinum treatment administered between the two electrodes of FIG. 7 approximately two days after treatment. Here, the attracting electrode was placed above the repellent electrode. The white areas indicate the presence of botulinum toxin by the toxin's ability to reduce perspiration, whereas the dark areas indicate the absence of toxin by the presence of perspiration. In addition to vertical movement upwards, lateral spreading along much the length of the attractive electrode was also observed.

FIG. 9 depicts a single substrate having an attractive electrode, a repellent electrode and a through gap between the two electrodes.

FIG. 10 depicts results of starch tests achieved using the electrode configuration of FIG. 9 approximately two days after injection of botulinum toxin. The white areas show where the toxin has reduced perspiration, and the dark areas indicate normal perspiration after exercise. Subject 1 (left); low power electrodes were used on right side of the face (left side of image). The test injection shows better spreading in the treatment area as well as migrating “up” toward the attractive electrode as compared to the negative control (no electrodes) injected in the same horizontal plane. Subject 2 (right); medium power electrodes used on left side of the face (right side of image). The test injection shows better spreading as well as migrating “down” toward the attractive electrode as compared to the negative control (no electrodes) injected in the same horizontal plane (the electrodes were reversed on the two subjects).

FIG. 11 shows follow up starch test results from Subject 1 (left) and Subject 2 (right) approximately 20 weeks after the study conducted with the electrode configuration of FIG. 9 confirming the localized toxin remained in its targeted position.

FIG. 12 depicts an exemplary device embodied as a headband.

FIG. 13 is an underside of a portion of the headband shown in FIG. 12 depicting repellent electrodes 30.

FIG. 14 is a table of botulinum toxins that can be administered in the disclosed methods.

### DETAILED DESCRIPTION

Described herein are devices and methods for directing dissipation of charged active agents after administration to a subject that not only improve the safety of procedures involving the administration of active agents such as neurotoxins but also improve spreading of the active agent at the intended treatment area, thereby improving the quality, uniformity and longevity of the procedure. The devices and methods are particularly useful to help prevent unwanted complications influenced by patient anatomy or when an understanding of the anatomy at the injection site or treatment site is limited, such as the complex nature of the skin and facial muscles around the eyes across different subjects. The devices and methods are also useful in instances where there is a risk of improper injection technique, such as by newly trained personnel or in instances where injections are difficult to properly administer.

In addition to improving the safety of procedures, it was surprisingly found that the devices and methods can also improve the quality of treatment and patient experience by creating more uniformity in therapeutic delivery over the treatment area. In particular, it was surprisingly found that in instances of applying neurotoxins during cosmetic procedures, the devices and methods can direct dissipation of the neurotoxin to more thoroughly “fill the gap” between and around injection sites, thereby reducing the number of injections needed for each treatment area.

The above benefits are accomplished by a technical approach of directing dissipation of an active agent after being administered to the subject in instances where the administered active agent carries a charge at physiological pH. Moreover, movement of the charged active agent is accomplished without piercing the skin. As a non-limiting example we show the ability to influence the movement of an administered charged active agent upwards, downwards, and laterally to more uniformly spread over a desired treatment area and to avoid spreading into regions where exposure is unwanted.

Further, the device operates using an adjustable but low power charge that does not cause patient discomfort and in fact, during testing was found to present less discomfort than the preceding administration of the active agent itself. Moreover, the adjustability of the power permits the device to be used across different subjects that may have different skin thicknesses, tensile strength, and sensitivities at the same or different areas of the body.

### Certain Terminology

Unless defined otherwise, all technical and scientific terms used herein have the same meaning as is commonly understood by one having ordinary skill in the medical, medical device, and pharmaceutical arts. It is also to be understood that the foregoing general description and detailed description are exemplary and explanatory only and are not restrictive of the any subject matter claimed.

“Active agent” means any compound that has biological or therapeutic activity. An “active agent” can be a biologically active component of a pharmaceutical composition. A “charged active agent” means a compound that has a biological or therapeutic activity and is charged at physiological pH.

“Administration,” or “to administer” means the step of giving (i.e. administering) a material such as a pharmaceutical composition or active agent or dermal filler to a subject. The materials disclosed herein can be administered via a number of appropriate routes, however as described in the disclosed methods, in embodiments the active agents are locally administered by e.g. intramuscular, intradermal, or subcutaneous routes of administration, such as by injection, topically, or use of an implant. Non-limiting examples include syringe/needle injection and iontophoresis.

“Applied to the skin” means an electrode is placed on top of the skin directly or on a substrate that is applied to the skin, which permits electric charge to sufficiently pass so that electric charge can reach a charged active agent that has been subcutaneously administered.

“Attractive electrode” means an electrode to which an attractive charge is applied in relation to the administered composition or active agent thereof (e.g. after dissociation at physiological pH). That is, an electrode with a positive charge or polarity would be an “attractive electrode” when used to direct dissipation of a composition or an active agent thereof having a net negative charge after administration.

“Attractive charge” means a charge that attracts the injected composition, an active agent of the injected composition (e.g. after dissociation from a complex at physiological pH), or a component of the injected material (e.g. charged ingredient). An example is the 150 kd neurotoxin component of the BoNT/A molecule, for example by use of an electric charge. An “attractive charge” has an opposite charge or polarity compared to the composition or active agent that is to be attracted.

“Botulinum toxin” or “botulinum neurotoxin” means a wild type neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa BoNT/A complexes) as well as pure botulinum toxin (i.e. the 150 kDa BoNT/A neurotoxic molecule), all of which are useful in the practice of the embodiments of the present disclosure.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Dermal filler” means compositions used for aesthetic treatments that are injected into or below the skin. Typically, they are designed to effectively reduce the appearance of unwanted wrinkles, contour and create volume, and to revitalize the skin. Suitable fillers can include hyaluronic acid, polyalkylimide, polylactic acid, Polymethyl-methacrylate microspheres (PMMA), and the like.

“Electrode” means an electrically conductive material that applies a charge to the subject to effect treatment. An “electrode” can be provided in any suitable pattern.

“Injection site” means the site where the injection or iontophoresis occurs.

“Flexible substrate” means the substrate can bend to follow the contour of the body or tissue where applied.

“Limiting” the dissipation of an administered material means that the composition, active agent (e.g. after dissociation of a complex), or a different component of the injected material is limited in its dissipation away from the intended treatment site so that the total area affected by the administered material is less than the total area would be in the absence of the limiting action.

“Localizing” as used herein means directing, increasing, or minimizing the dissipation of an active agent.

“Intermediate-acting” as used herein refers to a botulinum toxin that produces effects more slowly that a fast-acting toxin such as BoNT/E.

“Neurotoxin” means a biologically active molecule with a specific affinity for a neuronal cell surface receptor. “Neurotoxin” includes Clostridial toxins both as pure toxin and as complexed with one or more non-toxin, toxin-associated proteins, for example HN-33.

“Patient” or “subject” means a human or non-human mammal receiving medical or veterinary care.

“Parallel to the skin”, “approximately parallel to the skin”, and “substantially parallel to the skin” means the electric charge follows the path of the skin so that a charged active agent migrates along the path of the skin towards one of the electrodes when the electric field is generated. Thus, when the electric field is generated between opposing electrodes applied to the skin surface, the electric field can also be said to be “parallel to the skin”, “approximately parallel to the skin”, or “substantially parallel to the skin” within the dermis, epidermis or subcutaneous layer.

“Pharmaceutical composition” means a formulation that includes an active agent (e.g. neurotoxin). A “pharmaceutical composition” can be a dermal filler. The word “formulation” means that there is at least one additional ingredient, such as a pharmaceutically acceptable carrier or stabilizer to assist with delivery, (such as, for example and not limited to, an albumin (such as a human serum albumin or a recombinant human albumin) and/or sodium chloride) in the pharmaceutical composition in addition to the active agent. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Pharmaceutically acceptable carrier” refers to a substance that serves as a vehicle for improving the efficiency of delivery that is compatible with the other ingredients of the formulation and not substantially deleterious to the recipient thereof.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Repellent electrode” means an electrode to which a repellent charge is applied in relation to the composition, active agent, or component of the administered material for dissipation. That is, an electrode with a positive charge or polarity would be a repellent electrode when used to direct dissipation of an active agent having a net positive charge at physiological pH.

“Repellent charge” means a charge that repels the administered composition, active agent or component of the administered material, for example by way of a magnetic or electric charge. A “repellent charge” has a same charge or polarity as the composition, active agent or component of the material that is to be repelled

“Subcutaneous administration” as used herein comprises administration below the skin, which is intended to also include intramuscular administration.

“Substantially free” means present at a level of less than one percent by weight of a pharmaceutical composition or other material in which the weight percent of a substance is assessed.

“Therapeutic formulation” means a formulation that can be used to treat and thereby alleviate a disorder or a disease and/or symptom associated thereof, such as a disorder or a disease characterized by an activity of a peripheral muscle or nerve.

“Therapeutically effective amount” means the level, amount or concentration of an active agent (e.g. such as a botulinum toxin or pharmaceutical composition comprising botulinum toxin) needed to elicit a biological or medical response that is being sought. As to the electrodes, a “therapeutically effective amount” of charge or voltage is that which is sufficient to influence the position of the administered composition, active agent, or component of the administered material. A “therapeutically effective amount” will treat a disease, disorder or condition without causing significant negative or adverse side effects.

“Toxin-naïve” means a patient to whom a neurotoxin has not been administered, for example a Clostridial toxin, for example BoNT/A.

“Treat,” “treating,” or “treatment” means an alleviation or a reduction (which includes some reduction, a significant reduction a near total reduction, and a total reduction), resolution or prevention (temporarily or permanently) of a disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Treatment site” or “Treatment area” means the region of the body that the active agent is to be electrically delivered. The device directs dissipation to or within the “treatment area.” Examples of a “treatment site” or “treatment area” include an area(s) where wrinkles are present. In regard to migraine, the “treatment site” or “treatment area” is the location where the active agent is to be delivered to reduce migraine symptoms.

Unit” or “U” means an amount of active agent (e.g. BoNT) standardized to have equivalent neuromuscular blocking effect as a Unit of commercially available agent (e.g. BoNT/A).

### Dissipation of Active Agents

Described herein are devices, systems and methods for directing the dissipation of administered active agents through the use of energy applied to, above, around, or about the administration site and/or treatment site. Controlling the dissipation of active agents after administration can on the one hand prevent or reduce complications associated with the administration of active agents by “fencing off” particular biological areas where exposure to the active agent is unwanted and on the other hand improve therapeutic treatment by “fencing in” active agents at desired treatment areas or delivering active agents to desired treatment areas.

In some embodiments the systems, devices and methods are used to direct dissipation an administered neurotoxin away from an area where the dissipation of the neurotoxin is not desired, such as an area that can cause a complication (e.g. eyebrow ptosis/eyelid ptosis). In other embodiments, the systems, devices and methods are used to direct dissipation of an administered neurotoxin towards an intended treatment area (e.g. skin wrinkles). In still other embodiments, the systems, devices and methods are used to direct dissipation of an administered neurotoxin both away from a region where the presence of the neurotoxin is not desired and towards an intended treatment area.

Among the neurotoxins that the systems, devices and methods have particular utility are neurotoxins that carry a charge at physiological pH and which are used in treatments such as cosmetic procedures and in the treatment of migraine. For example, the 900 kd complex of BoNT/A has a published isoelectric point of roughly 9.2. Thus, at physiological pH values, BoNT/A carries a net positive charge. Our studies demonstrate that by applying a negative source charge to the skin, BoNT/A can be selectively dissipated toward a desired treatment site. Relatedly, by applying a positive source charge to the skin at a second location, for example around part or all of the perimeter of a treatment site, we were able to repel BoNT/A away from that second location. Thus, by applying a directional field that has both positive and negative sources we successfully migrated the charged composition to the desired treatment area and prevented migration in areas where the presence of BoNT/A was not desired. Moreover, we also surprisingly found that by increasing the length of the electrodes we were able to increase laterally spreading of the BoNT/A as it migrated towards the attractive electrode, thereby allowing improved sculpting of treatment over the entire treatment area.

As another non-limiting example, Type E (BoNT/E) has a published isoelectric point of about 6; therefore, at physiological pH values the toxin carries a net negative charge, and the “opposite” approach to that used with BoNT/A is appropriate, though conformational aspects of an administered material can affect its predicted isoelectric point, and thus ideal placement and strength of the forces applied can vary. The isoelectric point of the 150 kd neurotoxin component of BoNT/E has a published isoelectric point of roughly 6. Thus, at physiological pH values, the 150 kd neurotoxin component of BoNT/E carries a net negative charge. In embodiments, by applying a positive source charge to or around or near a location, for example a desired treatment or injection site, the 150 kd toxin can be “fixed” in or dissipation can be attracted to that location. Similarly, by applying a negative source charge to a second location, for example around part or all of the perimeter of a treatment site, the 150 kd toxin can be repelled or dissipated away from that second location.

One implication of the demonstrated ability to direct the dissipation of neurotoxins is that the systems, devices and methods can reduce the risk of unwanted effects or complications that can occur as a result of administration of the composition. For example, by directing the dissipation of botulinum toxins away from the eyes during cosmetic procedures, the incidence of brow ptosis, eyelid ptosis and other complications can be reduced. In addition, directing the dissipation of active agents to smaller treatment sites can reduce an adverse “immunogenic footprint”. For example, botulinum toxins are typically injected intramuscularly and bind rapidly. Without being bound by theory it is believed that not all of the material is bound immediately, and this material can dissipate from a treatment site to surrounding tissue, thereby increasing the odds of triggering an immunogenic reaction in the patient. It is believed the this component is highly affected by the devices and methods disclosed herein.

Directing the dissipation of can also increase the effect duration of administered materials themselves, for example the effect duration of a neurotoxin injection, or the effect duration of a dermal filler injection, thereby increasing the time duration before treatment is repeated.

Directing the dissipation of active agents can also increase the effect intensity of active agent, for example the effect intensity of a neurotoxin injection, or the effect intensity of a dermal filler injection.

Directing the dissipation of active agents can also accelerate the uptake of active agents, for example the uptake of a neurotoxin injection, by increasing the effective concentration of the material. Increased uptake can also be achieved by repeated contraction of a muscle in a treatment area to increase uptake while localization goals are achieved. Accordingly, in some embodiments the neurotoxin is electrically delivered to the desired treatment area using a first program, then the programming changes to deliver fast pulses at the attractive electrode to increase muscle contraction for increased uptake.

Directing the dissipation of the injected active agent can equilibrate the spread of the active agent over a desired treatment area, thus decreasing the amount of toxin required to achieve a desired effect. For example, “baby BOTOX” refers to an administration technique that utilizes minimal toxin amounts injected into a number of sites and is often used as a preventative measure among younger patients. However, as these treatments typically utilize administration via injection, disclosed embodiments can be used to direct dissipation throughout a treatment area such as the forehead to achieve a more equal distribution of the toxin while minimizing the number of injections required.

As will become more evident in the paragraphs that follow, although the disclosure primarily described with respect to directing the dissipation of neurotoxins, the systems, devices and methods can direct the dissipation of any charged composition, component of a composition, or component of an injected material that is positioned and able to move underneath the skin, such as at least up to a depth of few millimeters or so beneath the dermal layer. The suitability of a composition or component for dissipation can be assessed by its charge at physiological pH or measuring it's isoelectric point using techniques known in the art to which the invention belongs.

### Systems and Devices

Disclosed embodiments include systems and devices for directing, localizing, increasing, or minimizing the dissipation of charged active agents after administration, for example injected or iontophoresed pharmaceutical compositions. In some embodiments the systems and devices increase the spread or dissipation of administered active agent, for example an injected pharmaceutical composition. In other embodiments, the systems and devices decrease the spread or dissipation of active agents. The systems and devices operate by applying an energy field, for example an electromagnetic field (EMF) such as an electric field, an electric charge, an electric current, a magnetic field, or combinations thereof, to the outer skin, which forms a directional electric field in the dermal and subcutaneous layers, which is approximately parallel to the skin and thus directs dissipation of charged active agents towards one electrode and away from another. As non-limiting examples, dissipation can be directed for charged active agents including, for example, biologics, analgesics, anesthetics, neurotoxins, proteins, DNA, viruses, dermal fillers, and the like.

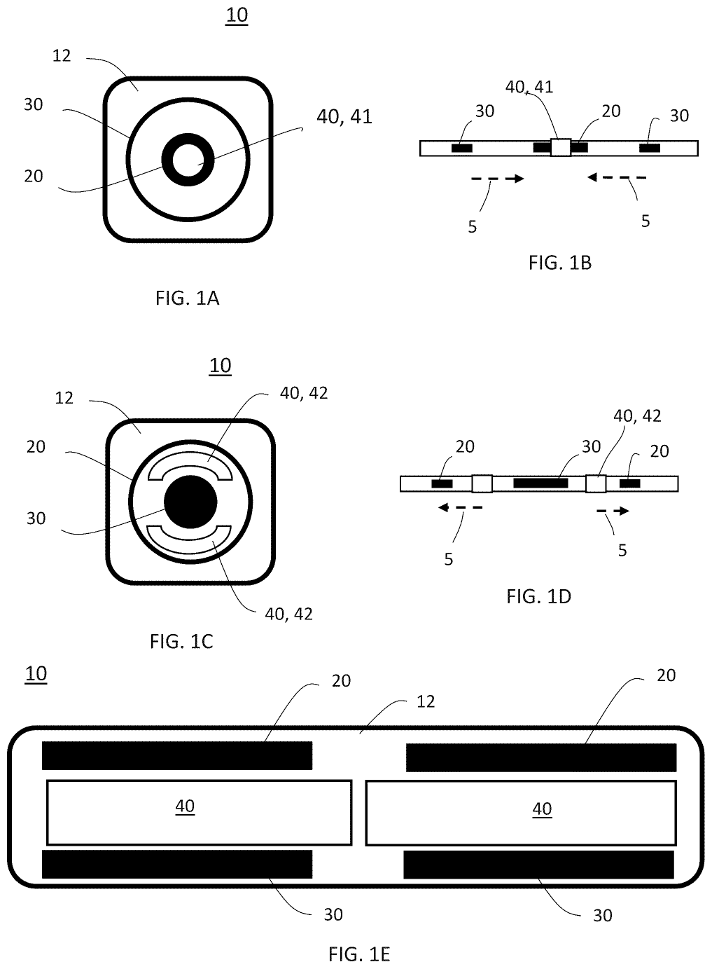

Turning now to FIGS. 1-6, an exemplary device 10 includes a flexible substrate 12, to which is applied two electrodes 20, 30 spaced apart from one another. One electrode 20, 30 is designated an attractive electrode 20 and the other a repellent electrode 30. The flexible substrate 12 is configured for placement against the skin of the subject where the electric charge or field is to be applied and preferably does not pierce the skin. A directional electric field is generated in the dermal and subcutaneous layers that extends approximately parallel to the skin between the electrodes. This electric charge acts on charged active agents to direct their dissipation towards the attractive electrode 20 and away from the repellent electrode 30.

### Electrodes

The electric charge is applied to the administered active agent by a flexible substrate 12 having at least two electrode 20, 30 of opposite charge or polarity secured to but preferably not penetrating the skin.

The substrate 12 can be formed from any suitable material having sufficiently flexibility for maintaining contact against the skin of the subject during operation and is preferably not electrically conductive so that the applied charge can be delivered solely through the electrodes 20, 30. As non-limiting examples, the substrate 12 can be formed from, a textile, fabric, polymer, or foam material to which the electrodes 20, 30 can be applied, such as by gluing electrically conductive wire or fiber. Electrodes 20, 30 can be sandwiched between substrate 12 layers, where a lower layer is preferably perforated to form a plurality of ports through which electric charge can traverse.

Referring more specifically now to FIGS. 1A and 1B, in some embodiments the device 10 is configured as a patch having an attractive electrode 20, a repellent electrode 30 extending at least partially around the perimeter of the attractive electrode 20, and a gap 40 in the form of a through bore 41, or port inside the attractive electrode 20 to allow for administration of a composition via iontophoresis or injection. The side view in FIG. 1B more clearly shows directional forces encouraging dissipation parallel to the skin 5 and towards the attractive electrode 20. As such, this configuration can be applied before or after administration of a active agent and would tend to direct dissipation so that the charged composition or components are “fenced in” or kept within the perimeter established by the repellent electrode 30.

FIGS. 1C and 1D also depict a device 10 configured as a patch but having the attractive electrode 20 extending at least partially around the perimeter of the repellent electrode 30, and a gap 40 in the form of a through slits 42 positioned between the attractive electrode 20 and the repellent electrode 30 to allow for administration of a composition via iontophoresis or injection. The side view in FIG. 1D more clearly shows directional forces encouraging dissipation parallel to the skin 5 and towards the attractive electrode 20. As such, this configuration can be applied before or after administration of an active agent and would tend to direct dissipation so that the charged active agent is dissipated outward towards the attractive electrode 20 and away from the repellent electrode 30.

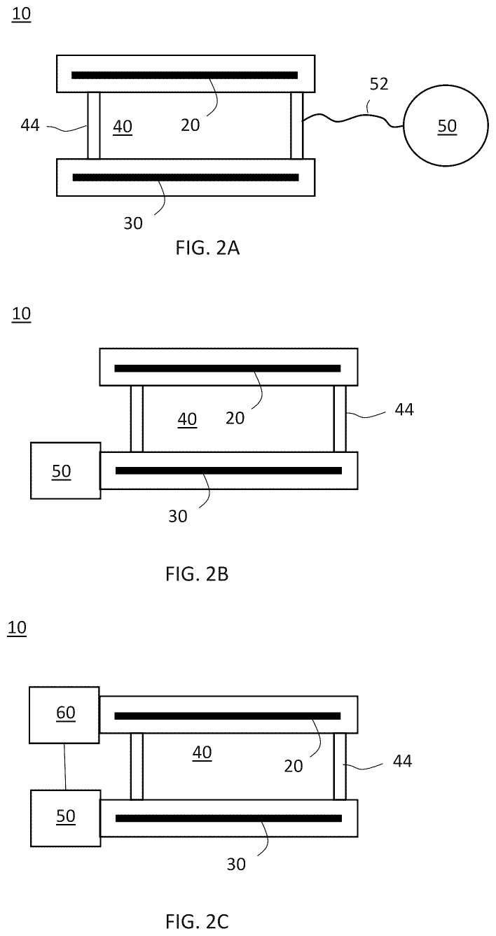

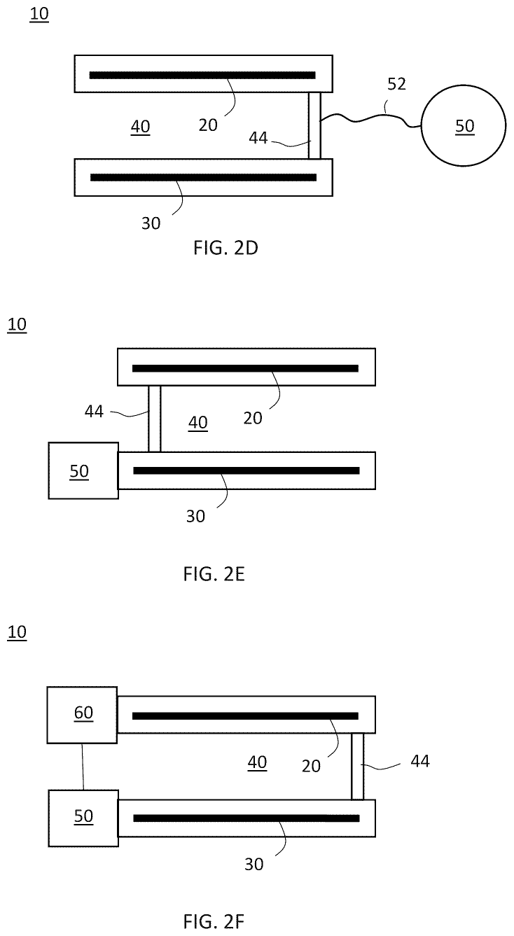

FIGS. 1E-2F provide different configurations where the device 10 has attractive electrodes 20 and repellent electrodes 30 substantially parallel to one another, and a gap 40 positioned between the attractive electrode 20 and the repellent electrode 30 to allow for administration of a composition via iontophoresis or injection before or after applying the device 10.

FIGS. 2A-2F provide a variation where attractive electrodes 20 and repellent electrodes 30 are tethered 44 to one another, thereby allowing electrodes 20, 30 to be spaced apart at variable distances for optimum effect and injection therebetween.

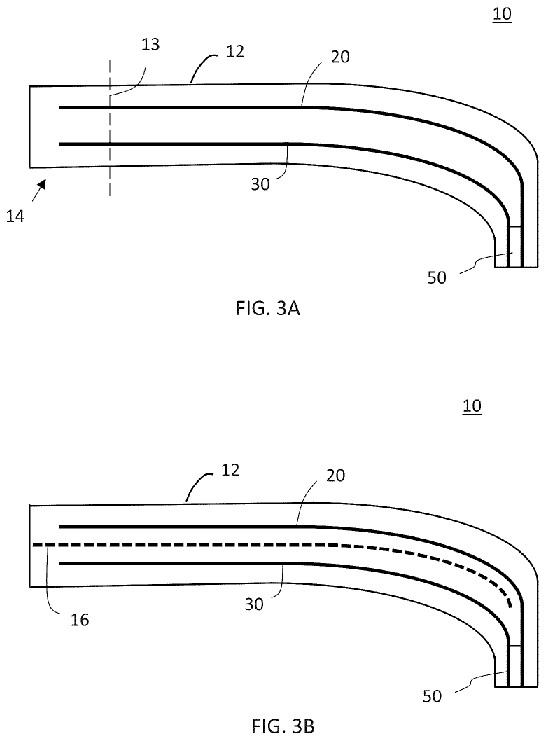

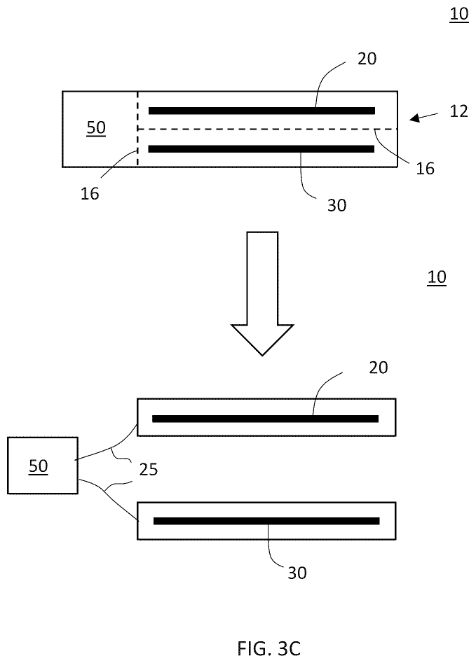

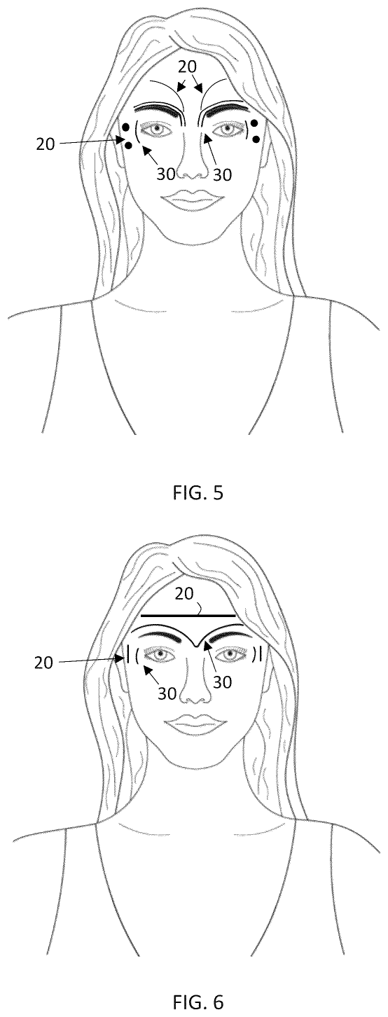

Moving on to FIGS. 3A-3C, in some embodiments the flexible substrate 12 is perforated 16 between the two electrodes 20, 30, thereby permitting the two electrodes 20, 30 to be pulled away from one another at the one end by tearing along the perforation 16. In FIGS. 3A-3B the substrate 12 is depicted in an arced embodiment intended to follow a path from a bridge of the nose and along the eyebrow. Here the flexibility of the substrate 12 permits the repellent electrode 30 to be placed close to the eyebrow to repel a charged composition away from the eyebrow and after torn along the perforation, the attractive electrode 20 can be placed higher on the subject's forehead (see also FIG. 5 and FIG. 6). This configuration is useful for the prevention of ptosis and is adjustable across a variety of patients. In various embodiments, the substrate 12 is at least 0.5, 1.0, or 1.5 inches in length but is usually shorter than 4 inches or so. Further, it has been surprisingly found that by increasing the length of the electrodes 20, 30 spreading of the charged active agent also tends to increase in length to follow the length of the electrodes 20, 30 and thus adjusting both the length (see FIG. 3A cut (13) and placement of the electrodes permits the treatment region to be more precisely sculpted.

Moving on to FIG. 3C, in some embodiments the flexible substrate 12 is perforated 16 between the two electrodes 20, 30, thereby permitting the two electrodes 20, 30 to be completely pulled away from one another by tearing along a perforation 16 so that they are tethered by a lead 25 connected to a battery 50.

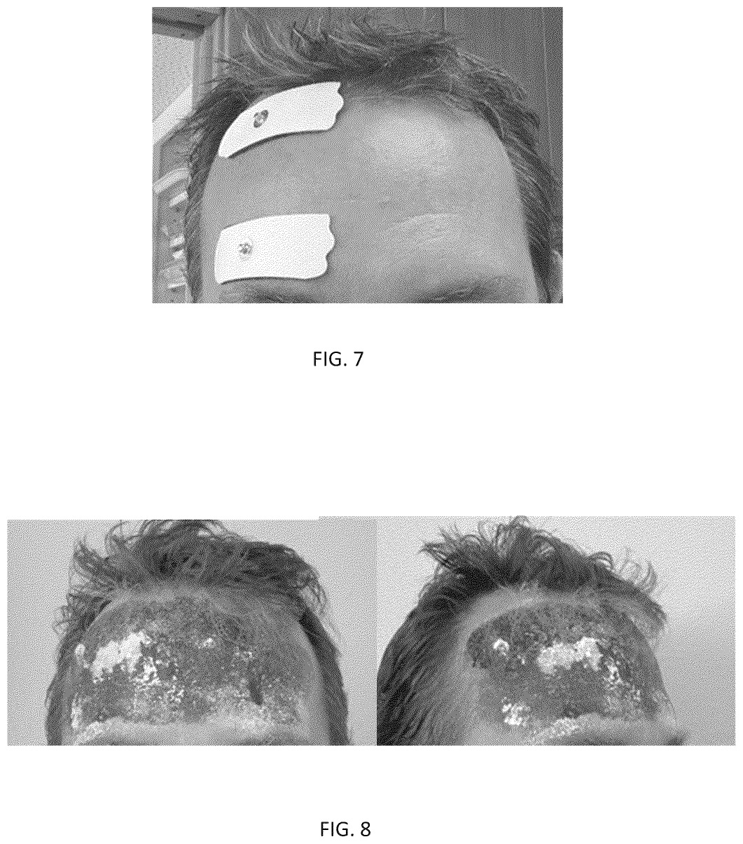

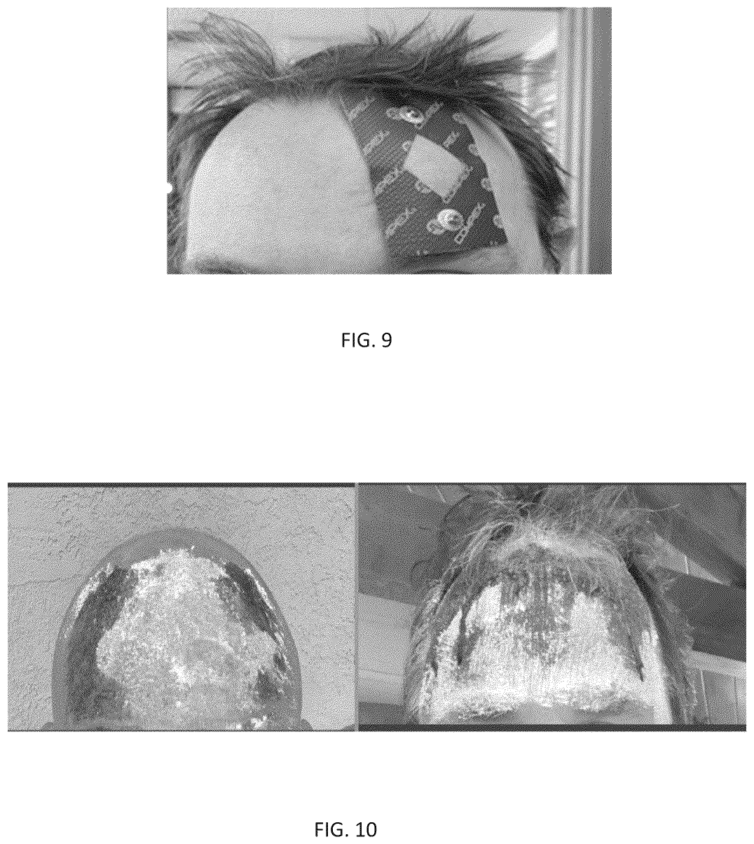

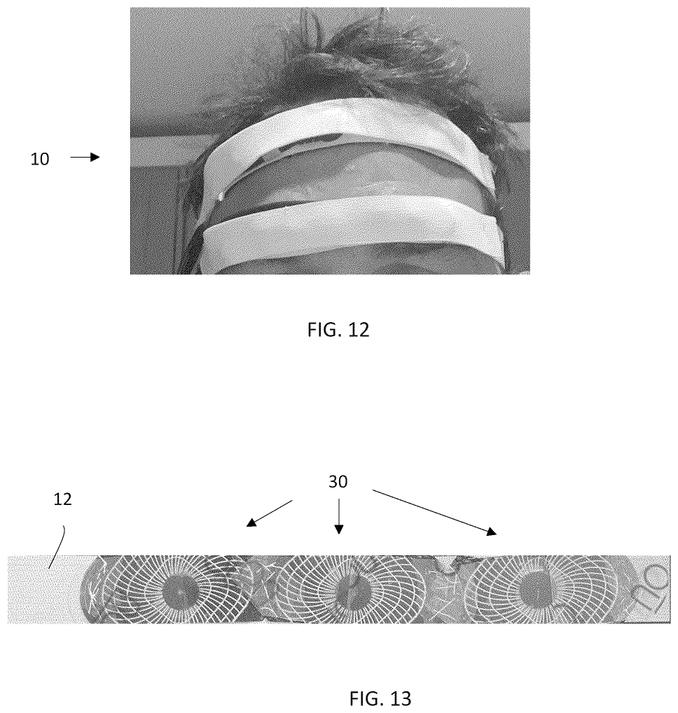

In any of the embodiments, the electrodes can be applied using an adhesive to maintain their position substantially as shown in FIGS. 7 and 9. Adhesives can be exposed by way of conventional peel away backings. In some embodiments, the substrate 12 is secured to the skin using a hydrogel as known the art to which the invention belongs. Alternatively, or in addition, the substrate 12 can be integrated into, for example, a headband. An example of a headband configuration is shown in FIG. 12. In other embodiments, the substrate 12 forms part of a mask or a wrap that at least partially presses against the subject during treatment. As shown more clearly in FIG. 13, preferably the electrodes 20, 30 are exposed on the underside of the substrate 12. Thus, the electrodes 20, 30 can be attached, for example reversibly, to a strap, mask, or headband to help maintain their position on the body. For example, in embodiments, a disclosed strap or headband comprises elastic or another stretchable material to secure the electrode(s) to the treatment area, for example around the head. Embodiments can comprise multiple straps, masks, or headbands, for example one strap, mask, or headband securing the attractive electrode 20 and another strap, mask, or headband securing the repellent electrode 30. Disclosed electrodes 20, 30 can include “snaps” or other connectors to reversibly attach the electrodes 20, 30 to a battery 50 or to a wire connected to the power supply 72. In embodiments, the electrodes comprise integral batteries.

In some embodiments, the device 10 comprises electrode connectors that differ in shape between the two electrodes 20, 30. For example, in embodiments, disclosed systems comprise lead connection points or electrode power connection points (collectively, “connectors”) that are non-generic. For example, in embodiments, a lead that connects the power supply 72 or controller to the electrode 20 comprising the attractive charge comprises a connector that enables connection of the lead to the attractive charge source or electrode 20, and not the repellent charge source or electrode 30, and vice versa. For example, leads can comprise mechanical shapes that differ in shape and render connection possible with only one of the attractive or repellent charge sources or electrodes 20, 30. In embodiments, such different shapes can comprise square shapes, round shapes, triangular shapes, and combinations thereof.

In embodiments, the electrodes 20, 30 can be reversibly attached to the strap, mask, or headband. In embodiments, the strap, mask, or headband can comprise holes or void regions, so as to provide a “pass-through” for connecting a power supply 20 to the electrodes 20, 30.

Further embodiments comprise devices 10 that require a power supply 72, for example a power supply 72 (or battery 50) connected to electrodes 20, 30 such that one or more electrodes 20, 30 produces a negative electric charge, and one or more electrodes 20, 30 produces a positive electric charge. Substrates 12 suitable for use with disclosed embodiments can comprise resorbable materials. Substrates 12 suitable for use with disclosed embodiments can comprise clear or opaque materials.

In embodiments, the power supply 72 comprises an energy source suitable for use with the body. For example, disclosed embodiments comprise use of a direct current (DC) energy source. In embodiments, the DC can comprise pulsed DC. In embodiments the power supply comprises a controller, for example to adjust power supply parameters such as intensity, duration, and the like. Further embodiments comprise a display, for example an LCD display or the like, to reflect power supply parameters such as intensity, duration, and the like. In embodiments, the power supply 72 and controller can be Bluetooth enabled to transmit data, such as power supply 72 parameters such as intensity, duration, and the like, to an additional device such as a smartphone.

In embodiments, the power supply 72 or battery 50 is connected to the electrodes 20, 30 via a wired connection.

Embodiments can also comprise integrated electrode systems wherein at least two electrodes 20, 30 are embedded into a substrate 12 that covers the entire treatment area. For example, a disclosed embodiment includes a flexible substrate that fits against a patient's head or a portion thereof, with the attractive and repellent electrodes 20, 30 placed to effectively apply a directional electric field across a treatment area, such as the forehead (see FIG. 9), the side of the head, the jaw, etc. In embodiments, the substrate 12 can comprise an injection “template”, with gaps 40, slits 42, or through bores 41 in the substrate where injections are to be made. For example, in disclosed embodiments, substrates 12 for positioning template electrodes 20, 30 can comprise voids, slots, or holes in locations typically used for neurotoxin injections, such as between the eyebrows for glabellar line treatments, or along the hairline for a forehead treatment. Such template embodiments can further comprise color coding and instructions for use.

In embodiments the flexible substrate 12 is clear or transparent, such that the electrodes 20, 30 are visible through the substrate 12, thus providing visual confirmation of exact electrode 20, 30 placement or active agent injection when applied to a patient.

By electrically coupling the power supply and electrodes, a disclosed system provides an energy, such as an electric field, to the body, for example the skin surface, which penetrates to the dermal and/or subcutaneous layers to form a directional electric field substantially parallel to the skin. In embodiments, an electric field can comprise voltages suitable for application to the mammal. For example, a suitable electric field can comprise voltages appropriate for non-lethal application to the body of a mammal, for example a human. In embodiments, the voltage can comprise sub-muscle contraction levels. In embodiments, the voltage can comprise levels that cause muscle contraction. For example, in embodiments that both localize administered materials as well as increase their uptake, muscle contraction can be beneficial.

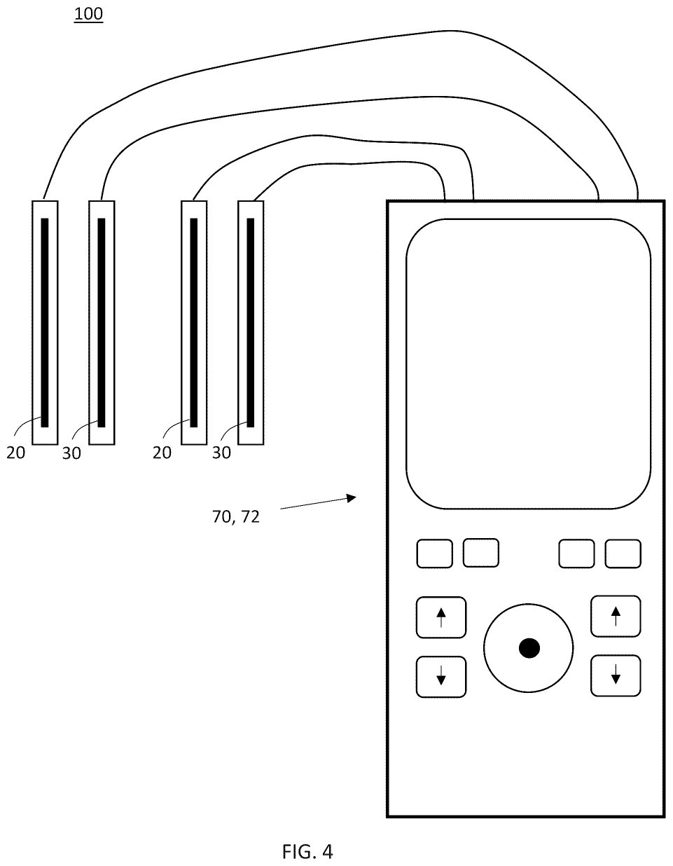

Disclosed embodiments comprise “active” devices that utilize a power source such as AC or DC power, or pulsed RF or pulsed current, such as high voltage pulsed current or pulsed DC, or “passive” devices that do not require external power. For example, in passive embodiments, the electrical energy can be derived from dissimilar metals creating a battery, for example wherein the dissimilar metals are located on a separate dressing or bandage, whereas those embodiments with an external power source can require conductive electrodes in a spaced-apart configuration to predetermine the electric field shape and strength. In active devices, DC current can be used. For example, as shown in FIGS. 2A-3B, electrodes 20, 30 can be powered by battery 50. In particular, FIGS. 2A and 2D depict configurations where a battery 50 is electrically connected to electrodes 20, 30 via lead 52. In such embodiments, battery 50 may be adhesively applied remote from the treatment site. In FIGS. 2B and 2D, battery 50 is attached to the substrate 12 and powers electrodes 20, 30. In FIGS. 2C and 2F, battery 50 is electrically connected to regulator 60 for regulating output to electrodes 20, 30. Configurations including regulator 60 may be desired when a same device 10 is configured for two or more different treatments or treatment locations utilizing different charge or voltage. Another embodiment is depicted in FIG. 4, where the electrodes 20, 30 are configured for connection to a EMS device 70, or a similar power supply 72 that can provide an appropriate charge or voltage to the skin.

Embodiments disclosed herein comprise bioelectric devices that comprise electrodes 20, 30. Such devices can include an electrode 20, 30 formed from a first conductive material, the material including a metal species; and a second electrode 20, 30 formed from a second conductive material, the material including a metal species capable of defining at least one voltaic cell for spontaneously generating at least one electrical current with the metal species of the first electrode 20, 30 when the first and second electrodes 20, 30 are connected via a conductive solution such as that within the body, and the first and second electrodes 20, 30 are not in physical contact with each other. Certain embodiments utilize an external power source such as AC or DC power or pulsed RF or pulsed current, such as high voltage pulsed current or pulsed AC or DC.

In embodiments, the attractive/repellent electric charge, field, or current is applied around, to, or about the treatment site. For example, in embodiments, electrodes 20, 30 are powered by, for example between 1 and 60V and applied about 1-3 cm apart, with the attractive electrode 20 applied upon or near the treatment site and the repellent electrode 30 applied to form a partial or complete perimeter around the treatment site so as to deter dissipation from the treatment site. The distance between the attractive and repellent electrodes 20, 30 can be determined based on the voltage applied to affect the most beneficial treatment. For example, the attractive and repellent electrodes 20, 30 can be applied 1 mm apart, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 16 mm apart, 17 mm apart, 18 mm apart, 19 mm apart, 2 cm apart, 2.5 cm apart, 3 cm apart, 3.5 cm apart, 4 cm apart, 4.5 cm apart, 5 cm apart, 5.5 cm apart, 6 cm apart, 6.5 cm apart, 7 cm apart, 7.5 cm apart, 8 cm apart, 8.5 cm apart, 9 cm apart, 10 cm apart, 10.5 cm apart, 11 cm apart, 11.5 cm apart, 12 cm apart, 12.5 cm apart, 13 cm apart, 13.5 cm apart, 14 cm apart, 14.5 cm apart, 15 cm apart, 15.5 cm apart, 16 cm apart, 16.5 cm apart, 17 cm apart, or more, or the like.

In embodiments, the distance between the attractive and repellent electrodes 20, 30 can be, for example, at least 1 mm apart, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 11 mm apart, at least 12 mm apart, at least 13 mm apart, at least 14 mm apart, at least 15 mm apart, at least 16 mm apart, at least 17 mm apart, at least 18 mm apart, at least 19 mm apart, at least 2 cm apart, at least 2.5 cm apart, at least 3 cm apart, at least 3.5 cm apart, at least 4 cm apart, at least 4.5 cm apart, at least 5 cm apart, at least 5.5 cm apart, at least 6 cm apart, at least 6.5 cm apart, at least 7 cm apart, at least 7.5 cm apart, at least 8 cm apart, at least 8.5 cm apart, at least 9 cm apart, at least 10 cm apart, at least 10.5 cm apart, at least 11 cm apart, at least 11.5 cm apart, at least 12 cm apart, at least 12.5 cm apart, at least 13 cm apart, at least 13.5 cm apart, at least 14 cm apart, at least 14.5 cm apart, at least 15 cm apart, at least 15.5 cm apart, at least 16 cm apart, at least 16.5 cm apart, at least 17 cm apart, or more, or the like.

In embodiments, the distance between the attractive and repellent electrodes 20, 30 can be, for example, not more than 1 mm apart, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 11 mm apart, not more than 12 mm apart, not more than 13 mm apart, not more than 14 mm apart, not more than 15 mm apart, not more than 16 mm apart, not more than 17 mm apart, not more than 18 mm apart, not more than 19 mm apart, not more than 2 cm apart, not more than 2.5 cm apart, not more than 3 cm apart, not more than 3.5 cm apart, not more than 4 cm apart, not more than 4.5 cm apart, not more than 5 cm apart, not more than 5.5 cm apart, not more than 6 cm apart, not more than 6.5 cm apart, not more than 7 cm apart, at not more than 7.5 cm apart, not more than 8 cm apart, not more than 8.5 cm apart, not more than 9 cm apart, not more than 10 cm apart, not more than 10.5 cm apart, not more than 11 cm apart, not more than 11.5 cm apart, not more than 12 cm apart, not more than 12.5 cm apart, not more than 13 cm apart, not more than 13.5 cm apart, not more than 14 cm apart, not more than 14.5 cm apart, not more than 15 cm apart, not more than 15.5 cm apart, not more than 16 cm apart, not more than 16.5 cm apart, not more than 17 cm apart, or more, or the like.

In embodiments, the distance between the attractive and repellent electrodes 20, 30 can be, for example, between 1 and 20 mm, between 2 and 18 mm, between 4 and 16 mm, between 6 and 14 mm, between 8 and 12 mm, or the like. In embodiments, the distance between the attractive and repellent electrodes 20, 30 can be, for example, between 1 and 10 mm, between 1 and 9 mm, between 1 and 8 mm, between 1 and 7 mm, between 1 and 6 mm, between 1 and 5 mm, between 1 and 4 mm, between 1 and 3 mm, between 1 and 2 mm, or the like. In embodiments, the distance between the attractive and repellent electrodes 20, 30 can be, for example, between 2 and 10 mm, between 2 and 9 mm, between 2 and 8 mm, between 2 and 7 mm, between 2 and 6 mm 2 and 5 mm, between 2 and 4 mm, between 2 and 3 mm, or the like.

In embodiments, the distance between the attractive and repellent electrodes 20, 30 can be, for example, between at least 1 and 20 mm, at least 2 and 18 mm, at least 4 and 16 mm, at least 6 and 14 mm, at least 8 and 12 mm, or the like. In embodiments, the distance between the attractive and repellent electrodes can be, for example, between at least 1 and 10 mm, at least 1 and 9 mm, at least 1 and 8 mm, at least 1 and 7 mm, at least 1 and 6 mm, at least 1 and 5 mm, at least 1 and 4 mm, at least 1 and 3 mm, at least 1 and 2 mm, or the like. In embodiments, the distance between the attractive and repellent electrodes can be, for example, between at least 2 and 10 mm, at least 2 and 9 mm, at least 2 and 8 mm, at least 2 and 7 mm, at least 2 and 6 mm, at least 2 and 5 mm, at least 2 and 4 mm, at least 2 and 3 mm, or the like.

In embodiments, the distance between the attractive and repellent electrodes 20, 30 can be, for example, between not more than 1 and 20 mm, not more than 2 and 18 mm, not more than 4 and 16 mm, not more than 6 and 14 mm, not more than 8 and 12 mm, or the like. In embodiments, the distance between the attractive and repellent electrodes 20, 30 can be, for example, between not more than 1 and 10 mm, not more than 1 and 9 mm, not more than 1 and 8 mm, not more than 1 and 7 mm, not more than 1 and 6 mm, not more than 1 and 5 mm, not more than 1 and 4 mm, not more than 1 and 3 mm, not more than 1 and 2 mm, or the like. In embodiments, the distance between the attractive and repellent electrodes 20, 30 can be, for example, between not more than 2 and 10 mm, not more than 2 and 9 mm, not more than 2 and 8 mm, not more than 2 and 7 mm, not more than 2 and 6 mm, not more than 2 and 5 mm, not more than 2 and 4 mm, not more than 2 and 3 mm, or the like.

In embodiments, disclosed devices 10 can comprise a circular or “donut” shaped device 10 (including a gap 40 or through bore 41 in the center) comprising an outer electrode 30 producing a repellent field, for example an electric field. For example, disclosed devices 10 can comprise an electrode 30 producing a repellent electric field that can be applied with the void or through bore 40 directly over the treatment site and the primary substrate 12 of the device 10 surrounding the treatment site. This type of embodiment can be particularly suitable for use with treatments where no spread of the injected material is desired. For example, in methods comprising administration of a neurotoxin to the patient's head, for example for treatment of migraine or depression or pain, disclosed devices 10 can be applied prior to the injections, then the injection is made through the void, through bore 40 or through slit 42 region.

In embodiments, disclosed devices 10 can comprise a circular or “donut” shaped device 10 comprising an electrode 30 producing a repellent field, for example an electric field, around the perimeter, while an electrode 20 producing an attractive field is in the center (See. FIG. 1A). This type of embodiment can be particularly suitable for use with treatments where no spread of the injected material is desired.

As shown in FIGS. 2A-4, the devices 10 can also be configured as elongated strips. Such configurations may be more preferred when a generally linear, repellent border is desired such as above the eyes during glabellar line treatment or during treatment of forehead lines. For example, in embodiments, a disclosed embodiment comprises linear electrodes 20, 30 placed above the eyebrows and below the hairline, for example to repel dissipation toward the eyes while increasing spread of the injected material throughout the forehead (see FIGS. 5-6).

In various embodiments the difference of the standard potentials of the electrodes 20, 30 providing attractive and repellent fields can be in a range from about 0.05 V to approximately about 10.0 V. For example, the standard potential can be about 0.05 V, about 0.06 V, about 0.07 V, about 0.08 V, about 0.09 V, about 0.1 V, about 0.2 V, about 0.3 V, about 0.4 V, about 0.5 V, about 0.6 V, about 0.7 V, about 0.8 V, about 0.9 V, about 1.0 V, about 1.1 V, about 1.2 V, about 1.3 V, about 1.4 V, about 1.5 V, about 1.6 V, about 1.7 V, about 1.8 V, about 1.9 V, about 2.0 V, about 2.1 V, about 2.2 V, about 2.3 V, about 2.4 V, about 2.5 V, about 2.6 V, about 2.7 V, about 2.8 V, about 2.9 V, about 3.0 V, about 3.1 V, about 3.2 V, about 3.3 V, about 3.4 V, about 3.5 V, about 3.6 V, about 3.7 V, about 3.8 V, about 3.9 V, about 4.0 V, about 4.1 V, about 4.2 V, about 4.3 V, about 4.4 V, about 4.5 V, about 4.6 V, about 4.7 V, about 4.8 V, about 4.9 V, about 5.0 V, about 5.1 V, about 5.2 V, about 5.3 V, about 5.4 V, about 5.5 V, about 5.6 V, about 5.7 V, about 5.8 V, about 5.9 V, about 6.0 V, about 6.1 V, about 6.2 V, about 6.3 V, about 6.4 V, about 6.5 V, about 6.6 V, about 6.7 V, about 6.8 V, about 6.9 V, about 7.0 V, about 7.1 V, about 7.2 V, about 7.3 V, about 7.4 V, about 7.5 V, about 7.6 V, about 7.7 V, about 7.8 V, about 7.9 V, about 8.0 V, about 8.1 V, about 8.2 V, about 8.3 V, about 8.4 V, about 8.5 V, about 8.6 V, about 8.7 V, about 8.8 V, about 8.9 V, about 9.0 V, about 10.0 V, about 11.0 V, about 12.0 V, about 13.0 V, about 14.0 V, about 15.0 V, about 16.0 V, about 17.0 V, about 18.0 V, about 19.0 V, about 20.0 V, about 21.0 V, about 22.0 V, about 23.0 V, about 24.0 V, about 25.0 V, about 26.0 V, about 27.0 V, about 28.0 V, about 28.0 V, about 30.0 V, about 31.0 V, about 32.0 V, about 33.0 V, about 34.0 V, about 35.0 V, about 36.0 V, about 37.0 V, about 38.0 V, about 39.0 V, about 40.0 V, or the like.

In embodiments, the electric field between the attractive and repellent electrodes 20, 30 can be, for example, 20 volt/cm, 19 volt/cm, 18 volt/cm, 17 volt/cm, 16 volt/cm, 15 volt/cm, 14 volt/cm, 13 volt/cm, 12 volt/cm, 11 volt/cm, 10 volt/cm, 9 volt/cm, 8 volt/cm, 7 volt/cm, 6 volt/cm, 5 volt/cm, 4 volt/cm, 3 volt/cm, 2 volt/cm, 1 volt/cm, or the like.

In embodiments, the electric field between the attractive and repellent electrodes 20, 30 can be, for example, between 20 volt/cm and 1 v/cm, between 19 volt/cm and 2 v/cm, between 18 volt/cm and 3 v/cm, between 17 volt/cm and 4 v/cm, between 16 volt/cm and 5 v/cm, between 15 volt/cm and 6 v/cm, between 14 volt/cm and 7 v/cm, between 13 volt/cm and 8 v/cm, between 12 volt/cm and 9 v/cm, between 11 volt/cm and 10 v/cm, or the like.

In embodiments, the electric field between the attractive and repellent electrodes 20, 30 can be, for example, no more than 20 volt/cm, no more than 19 volt/cm, no more than 18 volt/cm, no more than 17 volt/cm, no more than 16 volt/cm, no more than 15 volt/cm, no more than 14 volt/cm, no more than 13 volt/cm, no more than 12 volt/cm, no more than 11 volt/cm, no more than 10 volt/cm, no more than 9 volt/cm, no more than 8 volt/cm, no more than 7 volt/cm, no more than 6 volt/cm, no more than 5 volt/cm, no more than 4 volt/cm, no more than 3 volt/cm, no more than 2 volt/cm, no more than 1 volt/cm, or the like.

In embodiments, the electric field between the attractive and repellent electrodes 20, 30 can be, for example, no less than 20 volt/cm, no less than 19 volt/cm, no less than 18 volt/cm, no less than 17 volt/cm, no less than 16 volt/cm, no less than 15 volt/cm, no less than 14 volt/cm, no less than 13 volt/cm, no less than 12 volt/cm, no less than 11 volt/cm, no less than 10 volt/cm, no less than 9 volt/cm, no less than 8 volt/cm, no less than 7 volt/cm, no less than 6 volt/cm, no less than 5 volt/cm, no less than 4 volt/cm, no less than 3 volt/cm, no less than 2 volt/cm, no less than 1 volt/cm, or the like.

Embodiments can comprise an electric current between the attractive and repellent electrodes 20, 30. In embodiments, systems and devices disclosed herein can produce a low level electric current between the attractive and repellent electrodes 20, 30 of between for example about 1 and about 200 micro-amperes, between about 10 and about 190 micro-amperes, between about 20 and about 180 micro-amperes, between about 30 and about 170 micro-amperes, between about 40 and about 160 micro amperes, between about 50 and about 150 micro-amperes, between about 60 and about 140 micro-amperes, between about 70 and about 130 micro-amperes, between about 80 and about 120 micro-amperes, between about 90 and about 100 micro-amperes, between about 100 and about 150 micro-amperes, between about 150 and about 200 micro-amperes, between about 200 and about 250 micro-amperes, between about 250 and about 300 micro-amperes, between about 300 and about 350 micro-amperes, between about 350 and about 400 micro-amperes, between about 400 and about 450 micro-amperes, between about 450 and about 500 micro-amperes, between about 500 and about 550 micro-amperes, between about 550 and about 600 micro-amperes, between about 600 and about 650 micro-amperes, between about 650 and about 700 micro-amperes, between about 700 and about 750 micro-amperes, between about 750 and about 800 micro-amperes, between about 800 and about 850 micro-amperes, between about 850 and about 900 micro-amperes, between about 900 and about 950 micro-amperes, between about 950 and about 1000 micro-amperes (1 milli-amp [mA]), between about 1.0 and about 1.1 mA, between about 1.1 and about 1.2 mA, between about 1.2 and about 1.3 mA, between about 1.3 and about 1.4 mA, between about 1.4 and about 1.5 mA, between about 1.5 and about 1.6 mA, between about 1.6 and about 1.7 mA, between about 1.7 and about 1.8 mA, between about 1.8 and about 1.9 mA, between about 1.9 and about 2.0 mA, between about 2.0 and about 2.1 mA, between about 2.1 and about 2.2 mA, between about 2.2 and about 2.3 mA, between about 2.3 and about 2.4 mA, between about 2.4 and about 2.5 mA, between about 2.5 and about 2.6 mA, between about 2.6 and about 2.7 mA, between about 2.7 and about 2.8 mA, between about 2.8 and about 2.9 mA, between about 2.9 and about 3.0 mA, between about 3.0 and about 3.1 mA, between about 3.1 and about 3.2 mA, between about 3.2 and about 3.3 mA, between about 3.3 and about 3.4 mA, between about 3.4 and about 3.5 mA, between about 3.5 and about 3.6 mA, between about 3.6 and about 3.7 mA, between about 3.7 and about 3.8 mA, between about 3.8 and about 3.9 mA, between about 3.9 and about 4.0 mA, between about 4.0 and about 4.1 mA, between about 4.1 and about 4.2 mA, between about 4.2 and about 4.3 mA, between about 4.3 and about 4.4 mA, between about 4.4 and about 4.5 mA, between about 4.5 and about 5.0 mA, between about 5.0 and about 5.5 mA, between about 5.5 and about 6.0 mA, between about 6.0 and about 6.5 mA, between about 6.5 and about 7.0 mA, between about 7.5 and about 8.0 mA, between about 8.0 and about 8.5 mA, between about 8.5 and about 9.0 mA, between about 9.0 and about 9.5 mA, between about 9.5 and about 10.0 mA, between about 10.0 and about 10.5 mA, between about 10.5 and about 11.0 mA, between about 11.0 and about 11.5 mA, between about 11.5 and about 12.0 mA, between about 12.0 and about 12.5 mA, between about 12.5 and about 13.0 mA, between about 13.0 and about 13.5 mA, between about 13.5 and about 14.0 mA, between about 14.0 and about 14.5 mA, between about 14.5 and about 15.0 mA, between about 15 and about 20.0 mA, between about 20.0 and about 25.0 mA, between about 30.0 and about 35.0 mA, between about 40.0 and about 45.0 mA, between about 50.0 and about 55.0 mA, between about 60.0 and about 65.0 mA, or the like.

In embodiments, systems and devices disclosed herein can produce a low level electric current of between for example about 1 micro-ampere and about 1 milli-ampere, between about 50 and about 800 micro-amperes, between about 200 and about 600 microamperes, between about 400 and about 500 micro-amperes, or the like.

In embodiments, systems and devices disclosed herein can produce a low level electric current of about 10 micro-amperes, about 20 micro-amperes, about 30 microamperes, about 40 micro-amperes, about 50 micro-amperes, about 60 micro-amperes, about 70 micro-amperes, about 80 micro-amperes, about 90 micro-amperes, about 100 micro-amperes, about 110 micro-amperes, about 120 micro-amperes, about 130 microamperes, about 140 micro-amperes, about 150 micro-amperes, about 160 micro-amperes, about 170 micro-amperes, about 180 micro-amperes, about 190 micro-amperes, about 200 micro-amperes, about 210 micro-amperes, about 220 micro-amperes, about 240 microamperes, about 260 micro-amperes, about 280 micro-amperes, about 300 micro-amperes, about 320 micro-amperes, about 340 micro-amperes, about 360 micro-amperes, about 380 micro-amperes, about 400 micro-amperes, about 450 micro-amperes, about 500 microamperes, about 550 micro-amperes, about 600 micro-amperes, about 650 micro-amperes, about 700 micro-amperes, about 750 micro-amperes, about 800 micro-amperes, about 850 micro-amperes, about 900 micro-amperes, about 950 micro-amperes, about 1 milli-ampere (mA), about 1.1 mA, about 1.2 mA, about 1.3 mA, about 1.4 mA, about 1.5 mA, about 1.6 mA, about 1.7 mA, about 1.8 mA, about 1.9 mA, about 2.0 mA, about 2.1 mA, about 2.2 mA, about 2.3 mA, about 2.4 mA, about 2.5 mA, about 2.6 mA, about 2.7 mA, about 2.8 mA, about 2.9 mA, about 3.0 mA, about 3.1 mA, about 3.2 mA, about 3.3 mA, about 3.4 mA, about 3.5 mA, about 3.6 mA, about 3.7 mA, about 3.8 mA, about 3.9 mA, about 4.0 mA, about 4.1 mA, about 4.2 mA, about 4.3 mA, about 4.4 mA, about 4.5 mA, about 4.6 mA, about 4.7 mA, about 4.8 mA, about 4.9 mA, about 5.0 mA, about 5.1 mA, about 5.2 mA, about 5.3 mA, about 5.4 mA, about 5.5 mA, about 5.6 mA, about 5.7 mA, about 5.8 mA, about 5.9 mA, about 6.0 mA, about 6.1 mA, about 6.2 mA, about 6.3 mA, about 6.4 mA, about 6.5 mA, about 6.6 mA, about 6.7 mA, about 6.8 mA, about 6.9 mA, about 7.0 mA, about 7.1 mA, about 7.2 mA, about 7.3 mA, about 7.4 mA, about 7.5 mA, about 7.6 mA, about 7.7 mA, about 7.8 mA, about 7.9 mA, about 8.0 mA, about 8.1 mA, about 8.2 mA, about 8.3 mA, about 8.4 mA, about 8.5 mA, about 8.6 mA, about 8.7 mA, about 8.8 mA, about 8.9 mA, about 9.0 mA, about 9.1 mA, about 9.2 mA, about 9.3 mA, about 9.4 mA, about 9.5 mA, about 9.6 mA, about 9.7 mA, about 9.8 mA, about 9.9 mA, about 10.0 mA, about 10.1 mA, about 10.2 mA, about 10.3 mA, about 10.4 mA, about 10.5 mA, about 10.6 mA, about 10.7 mA, about 10.8 mA, about 10.9 mA, about 11.0 mA, about 11.1 mA, about 11.2 mA, about 11.3 mA, about 11.4 mA, about 11.5 mA, about 11.6 mA, about 11.7 mA, about 11.8 mA, about 11.9 mA, about 12.0 mA, about 12.1 mA, about 12.2 mA, about 12.3 mA, about 12.4 mA, about 12.5 mA, about 12.6 mA, about 12.7 mA, about 12.8 mA, about 12.9 mA, about 13.0 mA, about 13.1 mA, about 13.2 mA, about 13.3 mA, about 13.4 mA, about 13.5 mA, about 13.6 mA, about 13.7 mA, about 13.8 mA, about 13.9 mA, about 14.0 mA, about 14.1 mA, about 14.2 mA, about 14.3 mA, about 14.4 mA, about 14.5 mA, about 14.6 mA, about 14.7 mA, about 14.8 mA, about 14.9 mA, about 15.0 mA, about 15.1 mA, about 15.2 mA, about 15.3 mA, about 15.4 mA, about 15.5 mA, about 15.6 mA, about 15.7 mA, about 15.8 mA, about 16.0 mA, about 18.0 mA, about 20.0 mA, about 22.0 mA, about 24.0 mA, about 26.0 mA, about 28.0 mA, about 30.0 mA, about 32.0 mA, about 34.0 mA, about 36.0 mA, about 38.0 mA, about 40.0 mA, about 42.0 mA, about 44.0 mA, about 46.0 mA, about 48.0 mA, about 50.0 mA, or more, or the like.

In embodiments, the disclosed systems and devices can produce a low level electric current of not more than 10 micro-amperes, or not more than about 20 micro-amperes, not more than about 30 micro-amperes, not more than about 40 micro-amperes, not more than about 50 micro-amperes, not more than about 60 micro-amperes, not more than about 70 micro-amperes, not more than about 80 micro-amperes, not more than about 90 micro-amperes, not more than about 100 micro-amperes, not more than about 110 micro-amperes, not more than about 120 micro-amperes, not more than about 130 micro-amperes, not more than about 140 micro-amperes, not more than about 150 micro-amperes, not more than about 160 micro-amperes, not more than about 170 micro-amperes, not more than about 180 micro-amperes, not more than about 190 micro-amperes, not more than about 200 micro-amperes, not more than about 210 micro-amperes, not more than about 220 micro-amperes, not more than about 230 micro-amperes, not more than about 240 micro-amperes, not more than about 250 micro-amperes, not more than about 260 micro-amperes, not more than about 270 micro-amperes, not more than about 280 micro-amperes, not more than about 290 micro-amperes, not more than about 300 micro-amperes, not more than about 310 micro-amperes, not more than about 320 micro-amperes, not more than about 340 micro-amperes, not more than about 360 micro-amperes, not more than about 380 micro-amperes, not more than about 400 micro-amperes, not more than about 420 micro-amperes, not more than about 440 micro-amperes, not more than about 460 micro-amperes, not more than about 480 micro-amperes, not more than about 500 micro-amperes, not more than about 520 micro-amperes, not more than about 540 micro-amperes, not more than about 560 micro-amperes, not more than about 580 micro-amperes, not more than about 600 micro-amperes, not more than about 620 micro-amperes, not more than about 640 micro-amperes, not more than about 660 micro-amperes, not more than about 680 micro-amperes, not more than about 700 micro-amperes, not more than about 720 micro-amperes, not more than about 740 micro-amperes, not more than about 760 micro-amperes, not more than about 780 micro-amperes, not more than about 800 micro-amperes, not more than about 820 micro-amperes, not more than about 840 micro-amperes, not more than about 860 micro-amperes, not more than about 880 micro-amperes, not more than about 900 micro-amperes, not more than about 920 micro-amperes, not more than about 940 micro-amperes, not more than about 960 micro-amperes, not more than about 980 micro-amperes, not more than about 1 milli-ampere (mA), not more than about 1.1 mA, not more than about 1.2 mA, not more than about 1.3 mA, not more than about 1.4 mA, not more than about 1.5 mA, not more than about 1.6 mA, not more than about 1.7 mA, not more than about 1.8 mA, not more than about 1.9 mA, not more than about 2.0 mA, not more than about 2.1 mA, not more than about 2.2 mA, not more than about 2.3 mA, not more than about 2.4 mA, not more than about 2.5 mA, not more than about 2.6 mA, not more than about 2.7 mA, not more than about 2.8 mA, not more than about 2.9 mA, not more than about 3.0 mA, not more than about 3.1 mA, not more than about 3.2 mA, not more than about 3.3 mA, not more than about 3.4 mA, not more than about 3.5 mA, not more than about 3.6 mA, not more than about 3.7 mA, not more than about 3.8 mA, not more than about 3.9 mA, not more than about 4.0 mA, not more than about 4.1 mA, not more than about 4.2 mA, not more than about 4.3 mA, not more than about 4.4 mA, not more than about 4.5 mA, not more than about 4.6 mA, not more than about 4.7 mA, not more than about 4.8 mA, not more than about 4.9 mA, not more than about 5.0 mA, not more than about 5.1 mA, not more than about 5.2 mA, not more than about 5.3 mA, not more than about 5.4 mA, not more than about 5.5 mA, not more than about 5.6 mA, not more than about 5.7 mA, not more than about 5.8 mA, not more than about 5.9 mA, not more than about 6.0 mA, not more than about 6.1 mA, not more than about 6.2 mA, not more than about 6.3 mA, not more than about 6.4 mA, not more than about 6.5 mA, not more than about 6.6 mA, not more than about 6.7 mA, not more than about 6.8 mA, not more than about 6.9 mA, not more than about 7.0 mA, not more than about 7.1 mA, not more than about 7.2 mA, not more than about 7.3 mA, not more than about 7.4 mA, not more than about 7.5 mA, not more than about 7.6 mA, not more than about 7.7 mA, not more than about 7.8 mA, not more than about 7.9 mA, not more than about 8.0 mA, not more than about 8.1 mA, not more than about 8.2 mA, not more than about 8.3 mA, not more than about 8.4 mA, not more than about 8.5 mA, not more than about 8.6 mA, not more than about 8.7 mA, not more than about 8.8 mA, not more than about 8.9 mA, not more than about 9.0 mA, not more than about 9.1 mA, not more than about 9.2 mA, not more than about 9.3 mA, not more than about 9.4 mA, not more than about 9.5 mA, not more than about 9.6 mA, not more than about 9.7 mA, not more than about 9.8 mA, not more than about 9.9 mA, not more than about 10.0 mA, not more than about 10.1 mA, not more than about 10.2 mA, not more than about 10.3 mA, not more than about 10.4 mA, not more than about 10.5 mA, not more than about 10.6 mA, not more than about 10.7 mA, not more than about 10.8 mA, not more than about 10.9 mA, not more than about 11.0 mA, not more than about 11.1 mA, not more than about 11.2 mA, not more than about 11.3 mA, not more than about 11.4 mA, not more than about 11.5 mA, not more than about 11.6 mA, not more than about 11.7 mA, not more than about 11.8 mA, not more than about 11.9 mA, not more than about 12.0 mA, not more than about 12.1 mA, not more than about 12.2 mA, not more than about 12.3 mA, not more than about 12.4 mA, not more than about 12.5 mA, not more than about 12.6 mA, not more than about 12.7 mA, not more than about 12.8 mA, not more than about 12.9 mA, not more than about 13.0 mA, not more than about 13.1 mA, not more than about 13.2 mA, not more than about 13.3 mA, not more than about 13.4 mA, not more than about 13.5 mA, not more than about 13.6 mA, not more than about 13.7 mA, not more than about 13.8 mA, not more than about 13.9 mA, not more than about 14.0 mA, not more than about 14.1 mA, not more than about 14.2 mA, not more than about 14.3 mA, not more than about 14.4 mA, not more than about 14.5 mA, not more than about 14.6 mA, not more than about 14.7 mA, not more than about 14.8 mA, not more than about 14.9 mA, not more than about 15.0 mA, not more than about 15.1 mA, not more than about 15.2 mA, not more than about 15.3 mA, not more than about 15.4 mA, not more than about 15.5 mA, not more than about 15.6 mA, not more than about 15.7 mA, not more than about 15.8 mA, not more than about 16.0 mA, not more than about 18.0 mA, not more than about 20.0 mA, not more than about 22.0 mA, not more than about 24.0 mA, not more than about 26.0 mA, not more than about 28.0 mA, not more than about 30.0 mA, not more than about 32.0 mA, not more than about 34.0 mA, not more than about 36.0 mA, not more than about 38.0 mA, not more than about 40.0 mA, not more than about 42.0 mA, not more than about 44.0 mA, not more than about 46.0 mA, not more than about 48.0 mA, not more than about 50.0 mA, or more, or the like.

In embodiments, systems and devices disclosed herein can produce a low level electric current of not less than about 11.8 mA, not less than about 11.9 mA, not less than about 12.0 mA, not less than about 12.1 mA, not less than about 12.2 mA, not less than about 12.3 mA, not less than about 12.4 mA, not less than about 12.5 mA, not less than about 12.6 mA, not less than about 12.7 mA, not less than about 12.8 mA, not less than about 12.9 mA, not less than about 13.0 mA, not less than about 13.1 mA, not less than about 13.2 mA, not less than about 13.3 mA, not less than about 13.4 mA, not less than about 13.5 mA, not less than about 13.6 mA, not less than about 13.7 mA, not less than about 13.8 mA, not less than about 13.9 mA, not less than about 14.0 mA, not less than about 14.1 mA, not less than about 14.2 mA, not less than about 14.3 mA, not less than about 14.4 mA, not less than about 14.5 mA, not less than about 14.6 mA, not less than about 14.7 mA, not less than about 14.8 mA, not less than about 14.9 mA, not less than about 15.0 mA, not less than about 15.1 mA, not less than about 15.2 mA, not less than about 15.3 mA, not less than about 15.4 mA, not less than about 15.5 mA, not less than about 15.6 mA, not less than about 15.7 mA, not less than about 15.8 mA, not less than about 16.0 mA, not less than about 18.0 mA, not less than about 20.0 mA, not less than about 22.0 mA, not less than about 24.0 mA, not less than about 26.0 mA, not less than about 28.0 mA, not less than about 30.0 mA, not less than about 32.0 mA, not less than about 34.0 mA, not less than about 36.0 mA, not less than about 38.0 mA, not less than about 40.0 mA, not less than about 42.0 mA, not less than about 44.0 mA, not less than about 46.0 mA, not less than about 48.0 mA, not less than about 50.0 mA, or the like.

In embodiments, disclosed devices can provide an electric field of greater than physiological strength to a depth (as measured from the surface of the device) of, at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, or more.

In embodiments, disclosed devices can provide an electric field of greater than physiological strength to a depth (as measured from the surface of the device) of, not more than 1 mm, 2 mm, 3 mm, 4 mm, 5 mm, 6 mm, 7 mm, 8 mm, 9 mm, 10 mm, 1 1 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, or more.

In active embodiments, for example utilizing a powered supply 72 embodied as a TENS device, TENS can be applied at high frequency (>50 Hz) with an intensity below motor contraction (sensory intensity) or low frequency (<10 Hz) with an intensity that produces motor contraction. In embodiments, the attractive electrode 20 is placed atop the treatment area.

In further active embodiments, for example utilizing an EMS device 70, EMS 70 can be applied with an intensity below motor contraction or with minimal motor contraction. In embodiments, the attractive electrode 20 is placed atop the treatment area, or across the treatment area opposite or thereabouts from the repellent electrode.

In embodiments, an electric charge, field, current, or combinations thereof is applied using a system 100 comprising devices 10 comprising electrodes 20, 30. For example, a disclosed device 10 can comprise conductive wet or dry electrodes 20, 30. In embodiments, the device comprises attractive and repellent electrodes 20, 30, said attractive and repellent electrodes 20, 30 comprising opposing charges. For example, in powered devices 10, electrodes 20, 30 can comprise platinum or stainless steel, with one of the attractive/repellent electrodes 20, 30 comprising a positive charge, and the other comprising a negative charge.

Disclosed embodiments further comprise electrodes 20, 30 that can be used without skin preparation or the use of electrolytic gels. Disclosed electrodes 20, 30 can comprise at least one penetrator, which limits the depth of application, and/or anchors the electrode 20 30 or other device during normal application; and the use of stops which are integral with or separate from the penetrator that adjust the depth of application of the penetrator, and/or allows for uniform application of the electrode or other device over unprepared skin.

Disclosed embodiments comprise electrodes 20, 30 comprising a substrate, and at least one penetrator formed from a conductive material and protruding from the substrate wherein the penetrator is capable of piercing the stratum corneum or outer layer of the skin.

In embodiments, the size and shape of the penetrator is such that the penetrator(s) will not break or bend during normal use, will limit the depth the penetrator enters the skin under typical application conditions, and/or will anchor the electrode to prevent motion substantial movement. Therefore, preferably, the appropriate aspect ratio of the height to the average width of the penetrator, slope of the edge(s) or side(s) of the penetrator, and/or height of the penetrator are selected to make an electrode wherein the penetrator(s) will not break or bend, and will better anchor the electrode during application. The height of the penetrator(s) is measured from the tip of the penetrator perpendicular to the substrate. The penetrator(s), preferably, has a height from about 20 to about 150 μm, and more preferably from about 40 to about 100 μm. The aspect ratio of the penetrator is ratio of the height divided by the average width of the penetrator. The average width of the penetrator is measured by taking the widest average cross-section dimension of the cross-sections of the penetrator perpendicular to the height. The penetrator(s), preferably, has an aspect ratio of less than about 5, more preferably of less than about 2, even more preferably of less than about 1.5 and most preferably of less than about 0.75. The slope of the edge(s) or side(s) of the penetrator is measured by drawing a line tangent to the edge or the side of the penetrator(s) at any given point to the substrate 12 and measuring the angle between the line and where it intersects the upper surface 14 of the substrate. While it is understood that the slope may or may not vary substantially along the edge or side of the penetrator(s), preferably the slope is less than about 80 degrees over substantially all of the edge or side of the penetrator 16, more preferably is less than about 70 degrees, and most preferably is less than about 60 degrees. The penetrator(s) are used to push through the high impedance upper layer or stratum corneum of the epidermis to reduce the contact impedance of the electrode. Preferably, the penetrator(s) also “lock” the electrode into the chosen skin region.

Disclosed embodiments further comprise a EMS device 70 calibrated for use with disclosed methods. For example, disclosed EMS devices 70 comprise specific settings and electrodes suitable for use with specific muscles and neurotoxin formulations or brands. For example, a disclosed EMS device 70 as described herein comprises specific electrodes, voltages, and power settings designed to accommodate neurotoxin formulations. Thus, disclosed devices comprise electrodes 20, 30 suitable for narrow application of an electric field, for example for use as a repellent force in a glabellar line neurotoxin treatment, or larger electrodes, for example for use in treating larger muscles, or larger treatment areas.

Further embodiments comprise specific settings and electrodes 20, 30 suitable for use with specific treatment areas and dermal filler brands or formulations.

Disclosed power/controller device 70 embodiments comprise means for use, such as dials, buttons, a touchscreen, Bluetooth capability, a readable media slot, or the like to program the device to, for example, apply appropriate internal parameters for a BOTOX® treatment of the glabellar area, or a BOTOX® treatment of headache, or a DYSPORT® treatment of cervical dystonia, or a MYOBLOC® treatment of headache, a XEOMIN® treatment of hyperhidrosis, a dermal filler treatment of the cheeks, or a dermal filler treatment of the lips.

Thus, in embodiments, the disclosed power/controller device 70 allows the user to input the type, brand, or formulation of administered material and the treatment area, and the device will configure itself to apply preferred voltages or currents, as well as indicate to the user, for example visually, the appropriate electrodes to use with a specific treatment, and where to place the electrodes.

In further embodiments, a mechanical force can be applied to limit an injected material from dissipating. For example, in embodiments, a mechanical force such as that applied by an adhesive strip can be used to limit dissipation. In embodiments, the mechanical force can be applied in combination with an EMF force. In embodiments, the mechanical force can be applied with a clear adhesive tape or substrate.

Disclosed embodiments can comprise use of a system comprising a controller to provide and apply the voltage or current parameters for an optimal treatment. For example, in disclosed embodiments, the system 100 includes programming so that user is prompted to:
a. input a medical procedure such as a neurotoxin, dermal filler, or drug administration;
b. input the type or brand (or both) of neurotoxin or dermal filler or drug; and
c. input the area of treatment.

In disclosed embodiments, the system processes the input information, configures itself to supply the correct treatment voltages/currents or ranges thereof, and then directs the practitioner as to any or all of:
a. the appropriate treatment location or location range;
b. the appropriate injection location or location range;
c. the number of injections or range;
d. the appropriate material concentration and dosage or range;
e. the appropriate electrodes to employ; and
f. placement of the electrodes or range of placement.

In embodiments, photographs of the treatment area are taken, for example, both before, during, and after the procedure. In embodiments, the device “maps” the injection and electrode locations on to the photograph to assist the practitioner.

In embodiments a record of the administration can then be transmitted to the practitioner's record-keeping system, or a patient's phone, or the like. A “running” total of the patient's neurotoxin exposure can be maintained, in terms of units administered, total protein or dermal filler administered, treatment locations, and the like.

Further embodiments can comprise prompting the patient to submit an evaluation of the procedure to the practitioner, or a review of the service to a database, such as a consumer review database.

Further embodiments comprise user input to confirm that the procedure directed by the device (in terms of dosage, electrodes, voltages, etc.) was followed correctly. In embodiments this confirmation can then be supplied to a third party, for example the device or system manufacturer, a regulatory body, or an insurance organization.

Further embodiments comprise collection of data by a third party such as the device manufacturer, in order to track device performance and patient satisfaction.

Embodiments can include neurotoxin administrations to treat, for example, pain, muscle-related conditions including dystonia, spasticity and bladder conditions, depression, cosmetic concerns, and combinations thereof. For example, to treat pain, muscle-related conditions including dystonia, spasticity and bladder conditions, depression, cosmetic concerns, and the like, an attractive force, for example the attractive electrode (determined based upon the physical properties of an injected material, whether published or determined by experimentation) of, for example, a power/controller device can be placed on top of or in the vicinity of the injection site of a neurotoxin, with the repellent electrode of the device placed, for example, an inch or less from the attractive electrode. By means of electrodes disposed on but not penetrating the skin surface, electric flow is converted into an ionic current flow in the living tissue. The electrical current is generated and delivered across the intact skin surface via electrodes. In disclosed embodiments, the attractive electrode 20 can be placed over the injected material treatment site, or beyond the treatment site “opposite” the repellent electrode 30. In embodiments, the electrodes 20, 30 can be “wet” or “dry” electrodes. Wet electrodes 20, 30 typically use an electrolytic material as a conductor between the skin and the electrode. Dry electrodes typically consist of a single metal that acts as a conductor between the skin and the electrode.

Disclosed embodiments comprise methods of controlling release from “depot” drug administration. For example, an electric barrier can be established around a subcutaneous drug deposit, such as, for example, insulin or an analgesic.

Further disclosed embodiments can comprise articles of manufacture that include packaging material and an amount of a chemo-denervating agent, for example a neurotoxin. The chemo-denervating agent can be a Clostridial neurotoxin or a component thereof, for example a botulinum toxin such as botulinum toxin A (BoNT/A), botulinum toxin B (BoNT/B), botulinum toxin E (BoNT/E), botulinum toxin F (BoNT/F) or the neurotoxin components thereof, combinations thereof, and devices as disclosed herein. These articles of manufacture can comprise kits, for example comprising a neurotoxin or combinations thereof, disclosed devices, and instructions for use.

Disclosed embodiments comprise articles of manufacture that include packaging material and an amount of a cosmetic agent, for example a dermal filler such as hyaluronic acid (HA), and devices as disclosed herein. These articles of manufacture can comprise kits, for example comprising a dermal filler or fillers, disclosed systems and devices, and instructions for use.

Further embodiments can comprise a topical agent, for example a cream or gel comprising an attractive EMF, to apply to a treatment area following a treatment. For example, a cream that produces an attractive force can be applied over an injected material treatment site, such as the lips or the glabellar line area.

Further embodiments can comprise a topical agent, for example a cream or gel comprising a repellent EMF, to apply near a treatment area following a treatment. For example, a cream that produces a repellent force can be applied to limit dissipation of an injected material from a treatment site, such as the lips or the glabellar line area.

Further embodiments can comprise the use of a conductive material, for example a liquid, for example a hydrogel, between the electrodes and the skin surface, to lower the resistance of the skin. For example, resistance is the tendency of skin to impede electric current. Dry skin has a higher resistance than hydrated skin because the dry skin acts as a greater barrier to current flow. Most patients need between 15 and 50 milliamps (a milliamp is 1000th of an amp) for a strong but comfortable stimulation. Assuming the patient needs 25 milliamps and has healthy and well hydrated skin (and therefore a skin resistance of around 1000 ohms), then 25 volts is needed (25/1000×1000) to produce the desired current.

Embodiments comprise modified neurotoxins, said modification comprising increasing the inherent “charge” of the molecule, to increase the effect of disclosed embodiments.

Embodiments can also comprise adjusting the pH or conductivity of an injected material formulation.

Embodiments can also comprise adjusting the isoelectric point (pl) of an injected material or component thereof, for example using recombinant technologies, so as to make the material or component thereof more susceptible to manipulation via disclosed systems, devices, and methods.

### Methods of Use/Treatment

Embodiments comprise methods for directing dissipation of materials, for example topically applied, injected, or iontophoresed pharmaceutical compositions. Embodiments comprise the use of, for example, an energy field, for example an electromagnetic field (EMF) such as an electric field, an electric charge, an electric current, a magnetic field, or combinations thereof, to localize administered compositions. Administered compositions can comprise, for example, biologics, analgesics, anesthetics, biologics neurotoxins (both as complexes such as BoNT/A 900 kd, or the neurotoxin component alone, such as the 150 kd neurotoxin component of BoNT/A), proteins, DNA, viruses, dermal fillers, and the like. Localizing the injected materials can eliminate or reduce the spread or dissipation of the materials, thereby eliminating or reducing the risk of unwanted effects, as well as minimizing the “immunogenic footprint” that can result from an administration. Localizing the materials can increase the effect duration of administered materials, for example the effect duration of a neurotoxin injection, or the effect duration of a dermal filler injection, thereby increasing the time duration before treatment is repeated. Localizing the materials can increase the effect intensity of injected materials, for example the effect intensity of a neurotoxin injection, or the effect intensity of a dermal filler injection.

Embodiments comprise methods for increasing the spread or dissipation of active agents, for example injected charged active agents. Embodiments comprise the use of, for example, an energy field, for example an electromagnetic field (EMF) such as an electric field, an electric charge, an electric current, a magnetic field, or combinations thereof, to dissipate injected compositions. Injected compositions can comprise, for example, biologics, neurotoxins, proteins, DNA, viruses, dermal fillers, and the like.

Embodiments comprise methods for increasing the spread or dissipation of active agents, for example injected charged active agents. Embodiments comprise the use of, for example, an energy field, for example an electromagnetic field (EMF) such as an electric field, an electric charge, an electric current, a magnetic field, or combinations thereof, to localize injected compositions. Injected compositions can comprise, for example, biologics, neurotoxins, proteins, DNA, viruses, dermal fillers, and the like.

In embodiments, the attractive and repellent forces are applied to form an uninterrupted perimeter around a treatment area.

In embodiments, the attractive and repellent forces are applied to form a discontinuous perimeter around a treatment area.

In embodiments, the attractive and repellent forces are applied parallel to each other around a treatment area.

In embodiments, the attractive force is placed on top of a treatment area, while the repellent force is applied away from the treatment area to reduce or prevent active agent dissipation away from the treatment area.

In embodiments, the repellent force is placed on top of a treatment area, while the attractive force is applied away from the treatment area to increase directional active agent dissipation away from a sensitive treatment area.

In embodiments, the attractive or repellent force is placed beyond a treatment area opposite the corresponding opposite force, such that the force is exerted across the treatment area.

In disclosed embodiments of treatment, a material comprising a charged active agent is injected subcutaneously to, for example, between 1 and 15 mm tissue depth, then an attractive force comprising an electric charge is applied to, around, or partially around the treatment site. In particular, an electrode 20 placed on but not penetrating the skin applies the attractive force. The order of this operation can be reversed, such that the force is applied before, during, or after the administration; however, most preferably the electrode 20 is at least applied to the skin before injection to avoid pressing against the treatment site after injection.

In disclosed embodiments of treatment, a material comprising a charged active agent is injected subcutaneously to, for example, between 1 and 15 mm tissue depth, then a repellent force comprising an electric charge is applied around or partially around the treatment site, to “shield” an area where dissipation is to be avoided or limited. In particular, an electrode 30 placed on but not penetrating the skin applies the repellent force. The order of this operation can be reversed, such that the force is applied before, during, or after the administration; however, most preferably the electrode 30 is at least applied to the skin before injection to avoid pressing nearby the treatment site after injection.

In disclosed embodiments of treatment, a material comprising a charged active agent is injected subcutaneously to, for example, between 1 and 15 mm tissue depth, then an attractive force comprising an electric charge is applied to, around, or partially around the treatment site, and a repellent force comprising an electric charge is applied around or partially around the treatment site, wherein the attractive and repellent forces comprise opposite electric charges. In particular, electrodes 20, 30 are placed on but not penetrating the skin to apply the attractive and repellent forces. The order of this operation can be reversed, such that the force is applied before or after the administration; however, most preferably the electrodes 20, 30 are at least applied to the skin before injection to avoid pressing nearby the treatment site after injection. In embodiments, the attractive and repellent forces establish an electric field. In embodiments wherein there is electrical conductivity between the attractive and repellent forces, an electric current can be established.

In disclosed methods of treatment, localizing the injected charged active agent can comprise applying an energy field, for example an electric charge, field or current, or combinations thereof, to at least one injection site, or applying an energy field, for example an energy field, for example an electric charge, field or current, or combinations thereof, surrounding at least one injection site, and combinations thereof. For example, an energy field, for example an attractive electric charge, field or current, or combinations thereof, can be applied directly on top of the injection site. In embodiments, an energy field, for example a repellent electric charge, field or current, or combinations thereof, can be applied around the perimeter of one or multiple injection sites. These methods can be combined such that attractive and repellent forces are applied.

In embodiments, methods of minimizing an immune response caused by injected materials comprising a charged active agent can comprise applying an energy field, for example an electric charge, field or current, or combinations thereof, to at least one injection site, or surrounding at least one injection site. For example, an attractive energy field, for example an electric charge, field or current, or combinations thereof, can be applied directly on top of the injection site. In embodiments, a repellent energy field, for example an electric charge, field or current, or combinations thereof, can be applied around the perimeter or a part thereof of one or multiple injection sites. These methods can be combined such that attractive and repellent fields are applied.

In disclosed methods the energy field can comprise, for example, an electric field, electric charge, electric current, a magnetic field, a combination thereof, or the like. For example, an electric field can be applied to an injection site through the use of an electrode supplying a suitable voltage to produce an attractive field to “attract” the materials. In embodiments, an electric field can be applied to surround (either partially or completely) an injection site through the use of an electrode supplying a suitable voltage to produce a repellent field to “repel” the materials. In embodiments, both attractive and repellent field are applied. In this manner, the spread or dissipation of the injected materials can be reduced or eliminated.

For example, treatment of migraine in disclosed embodiments can comprise neurotoxin injections into at least one of the following muscles comprising the corrugator, the procerus, the frontalis, the temporalis, the occipitalis, the cervical paraspinal muscles, and the trapezius. In certain embodiments, at least one of the corrugator, the procerus, the frontalis, the temporalis, the occipitalis, the cervical paraspinal muscles, and the trapezius are specifically excluded from injection. In disclosed embodiments, the treatment involves 31 injections of 5 units per injection, for a total of 155 units.

In migraine treatment embodiments comprising injection of the corrugator, 5 units can be injected into each corrugator muscle. In migraine treatment embodiments comprising injection of the procerus, 5 units can be injected into the muscle. In migraine treatment embodiments comprising injection of the frontalis, 5 units can be injected into each of 4 sites in the muscle. In migraine treatment embodiments comprising injection of the temporalis, 5 units can be injected into each of 8 sites in the muscle. In migraine treatment embodiments comprising injection of the occipitalis, 5 units can be injected into each of 8 sites in the muscle. In migraine treatment embodiments comprising injection of the cervical paraspinal, 5 units can be injected into each of 4 sites in the muscle. In migraine treatment embodiments comprising injection of the trapezius, 5 units can be injected into each of 6 sites in the muscle.

Disclosed methods of treatment can comprise treating the indications of Table 1:

TABLE 1

| | | | | |
|---|---|---|---|---|
| Cervical dystonia | Lower limb | Urinary | Hyperhidrosis | Anismus |
| | spasticity | incontinence | | |
| Migraine | Upper limb | Overactive | Anal fissure | Atrial flutter |
| | spasticity | bladder | | |
| Blepharospasm | Achalasia | Alopecia | | Autonomic |
| | | | | dysreflexia |
| Lower limb | Dysphonia | Neuromyotonia | Rhinorrhoea | Benign |
| spasticity | | | and/or rhinitis | prostatic |
| | | | | hyperplasia |
| Strabismus | Endometriosis | Nystagmus | Sialorrhea | Bruxism |
| Glabellar lines | Oscillopsia | Esophageal | Facial flushing | Carpal tunnel |
| | | spasm | | syndrome |
| Obesity | Spasmodic | Exotropia, | Fecal | Osteoarthritis |
| | dysphonia | esotropia, | incontinence | |
| | | entropion | | |
| Orbital atrophy | Stiff person | Eyelid-opening | Frey's syndrome | Some forms |
| | syndrome | apraxia | | of pain |
| Stuttering | Tension | Piriformis | Hemifacial | Cleft lip |
| | headache | syndrome | spasm | repair |
| Synkinesis | Tetanus | Plantar fasciitis | Hyperlacrimation | Club foot |
| Temporomandibular | Tremor | Protective | Lateral | Constipation |
| joint syndrome | | ptosis | epicondylalgia | |
| Tennis elbow | Trigeminal | Psoriasis | Myofascial pain | Cystitis |
| | neuralgia | | | |
| Gastroparesis | Palatal | Vaginismus | Diabetic | Depression |
| | myoclonus | | polyneuropathy | |
| Gustatory sweating | Paratonia | Ventricular | Peyronie's | Vocal tics |
| | | arrhythmias | syndrome | |
| Restless leg | Myokymia | | | |
| syndrome | | | | |

Disclosed methods comprise treatment of the bladder comprising placing a cystoscope into the bladder and injecting a neurotoxin into numerous sites in via a needle that fits through the cystoscope.

Disclosed hyperhidrosis treatments can comprise administration of, for example, 100 U of BoNT/A reconstituted with 2.5 mL of 0.9% sterile saline subdivided into 25 subdermal injections.

Disclosed depression treatment methods can comprise applying attractive and repellent forces to the surface of the skin without physically penetrating the skin with electrodes; and injecting neurotoxin into the corrugators, orbicularis oculi, and the frontalis muscle.

Disclosed frown line treatments can comprise applying attractive and repellent forces to the surface of the skin without physically penetrating the skin with electrodes; and injecting a neurotoxin into the corrugator and procerus.

Disclosed horizontal forehead lines treatments can comprise applying attractive and repellent forces to the surface of the skin without physically penetrating the skin with electrodes; and injecting a neurotoxin into the frontalis. (see FIG. 7 and FIG. 9)

Disclosed crow's feet treatments can comprise applying attractive and repellent forces to the surface of the skin without physically penetrating the skin with electrodes; and injecting a neurotoxin into the orbicularis (see FIG. 5 and FIG. 6).

Disclosed bunny line treatments can comprise applying attractive and repellent forces to the surface of the skin without physically penetrating the skin with electrodes; and injecting a neurotoxin or dermal filler into the nasalis.

Disclosed radial lip line treatments can comprise applying attractive and repellent forces to the surface of the skin without physically penetrating the skin with electrodes; and injecting a neurotoxin or dermal filler into the orbicularis.

Disclosed Marionette line treatments can comprise applying attractive and repellent forces to the surface of the skin without physically penetrating the skin with electrodes; and injecting a neurotoxin or dermal filler into the depressor anguli.

Disclosed “gummy smile” treatments can comprise applying attractive and repellent forces to the surface of the skin without physically penetrating the skin with electrodes; and injecting a neurotoxin or dermal filler into the levator labii superioris.

Disclosed chin line treatments can comprise applying attractive and repellent forces to the surface of the skin without physically penetrating the skin with electrodes; and injecting a neurotoxin or dermal filler into the mentalis.

In embodiments, disclosed methods can comprise neurotoxin administration. For example, disclosed methods can comprise applying attractive and repellent forces to the surface of the skin without physically penetrating the skin with electrodes; and administration of neurotoxins such as BOTOX®, DYSPORT®, MYOBLOC®, XEOMING, JEUVEAU®, combinations thereof, and the like.

Disclosed treatment methods comprising BOTOX® administration can comprise treatment of urinary incontinence due to detrusor overactivity associated with a neurologic condition [e.g., spinal cord injury (SCI), multiple sclerosis (MS)] in adults who have an inadequate response to or are intolerant of an anticholinergic medication, prophylaxis of headaches in adult patients with chronic migraine (≥15 days per month with headache lasting 4 hours a day or longer), temporary improvement in the appearance of moderate to severe glabellar lines associated with procerus and corrugator muscle activity in adults <65 years of age, treatment of upper limb spasticity in adult patients, treatment of cervical dystonia in adult patients, to reduce the severity of abnormal head position and neck pain, treatment of severe axillary hyperhidrosis that is inadequately managed by topical agents in adult patients, treatment of blepharospasm associated with dystonia in patients ≥12 years of age, treatment of strabismus in patients ≥12 years of age, treatment of facial wrinkles, and the like.

Disclosed treatment methods comprising DYSPORT® can comprise treatment of cervical dystonia in adults, temporary improvement in the appearance of moderate to severe glabellar lines associated with procerus and corrugator muscle activity in adults <65 years of age, treatment of upper and lower limb spasticity in adults, treatment of upper limb spasticity in pediatric patients 2 years of age and older, excluding spasticity caused by cerebral palsy, treatment of lower limb spasticity in pediatric patients 2 years of age and older, and the like.

Disclosed treatment methods comprising XEOMIN® can comprise treatment of chronic sialorrhea, upper limb spasticity, cervical dystonia, blepharospasm, temporary improvement in the appearance of moderate to severe glabellar lines with corrugator and/or procerus muscle activity, and the like.

Disclosed treatment methods comprising MYOBLOC® can comprise treatment of cervical dystonia to reduce the severity of abnormal head position and neck pain associated with cervical dystonia in adults, treatment of chronic sialorrhea in adults, and the like.

Disclosed treatment methods can comprise avoidance of adverse effects, for example avoidance of neck pain, headache, worsening migraine, muscular weakness, dysphagia, and eyelid ptosis.

Disclosed treatment methods can comprise extended duration of effect as compared to methods known in the art. For example, disclosed embodiments can comprise an extended duration of effect of at least 5% greater, at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, or more, or the like.

Disclosed treatment methods can comprise accelerated onset of effect as compared to methods known in the art. For example, disclosed embodiments can comprise an accelerated onset of effect of at least 5% greater, at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, or more, or the like.

Disclosed treatment methods can comprise increased intensity of effect as compared to methods known in the art. For example, disclosed embodiments can comprise an increased intensity of effect of at least 5% greater, at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, or more, or the like.

Disclosed treatment methods can comprise lessening the immunogenic effect as compared to methods known in the art. For example, disclosed embodiments can comprise an immunogenic effect of at least 5% less, at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, at least 100% less, or more, or the like.

Disclosed treatment methods can comprise lessening the required dosage to achieve the desired effect as compared to methods know in the art. For example, disclosed embodiments can comprise a dosage of at least 5% less, a dosage of at least 10% less, a dosage of at least 15% less, a dosage of at least 20% less, a dosage of at least 25% less, a dosage of at least 30% less, a dosage of at least 35% less, a dosage of at least 40% less, a dosage of at least 45% less, a dosage of at least 50% less, a dosage of at least 55% less, a dosage of at least 60% less, a dosage of at least 65% less, a dosage of at least 70% less, a dosage of at least 75% less, a dosage of at least 80% less, a dosage of at least 85% less, a dosage of at least 90% less, a dosage of at least 95% less, or the like.

In disclosed embodiments, the administered material is formulated in unit dosage form; for example, a neurotoxin can be provided as a sterile solution in a vial or as a vial or sachet containing a lyophilized powder for reconstituting in a suitable vehicle such as saline for injection. Although examples of routes of administration and dosages are provided, the appropriate route of administration and dosage are generally determined on a case by case basis by the attending physician. Such determinations are routine to one of ordinary skill in the art. For example, the route and dosage for administration of a Clostridial neurotoxin according to the present disclosure can be selected based upon criteria such as the solubility characteristics of the neurotoxin chosen as well as the intensity and scope of the condition being treated.

In embodiments, the neurotoxin can be administered in an amount of between about 10 U/kg and about 35 U/kg. In an embodiment, the neurotoxin is administered in an amount of between about 10 U/kg and about 25 U/kg. In another embodiment, the neurotoxin is administered in an amount of between about 10 U/kg and about 15 U/kg. In another embodiment, the neurotoxin is administered in an amount of between about 1 U/kg and about 10 U/kg. In many instances, an administration of from about 1 unit to about 500 units of a neurotoxin, such as a botulinum type E, provides effective therapeutic relief. In an embodiment, from about 5 units to about 200 units of a neurotoxin, such as a botulinum type E, can be used and in another embodiment, from about 10 units to about 100 units of a neurotoxin, such as a botulinum type E, can be locally administered into a target tissue such as a muscle.

In embodiments, administration can comprise a dose of about 10 units of a neurotoxin, or about 20 units of a neurotoxin, or about 30 units of a neurotoxin, or about 40 units of a neurotoxin, or about 50 units of a neurotoxin, or about 60 units of a neurotoxin, or about 70 units of a neurotoxin, or about 80 units of a neurotoxin, or about 90 units of a neurotoxin, or about 100 units of a neurotoxin, or about 1 10 units of a neurotoxin, or about 120 units of a neurotoxin, or about 130 units of a neurotoxin, or about 140 units of a neurotoxin, or about 150 units of a neurotoxin, or about 160 units of a neurotoxin, or about 170 units of a neurotoxin, or about 180 units of a neurotoxin, or about 190 units of a neurotoxin, or about 200 units of a neurotoxin, or about 210 units of a neurotoxin, or about 220 units of a neurotoxin, or about 230 units of a neurotoxin, or about 240 units of a neurotoxin, or about 250 units of a neurotoxin, or about 260 units of a neurotoxin, or about 270 units of a neurotoxin, or about 280 units of a neurotoxin, or about 290 units of a neurotoxin, or about 290 units of a neurotoxin, or about 300 units of a neurotoxin, or about 310 units of a neurotoxin, or about 320 units of a neurotoxin, or about 330 units of a neurotoxin, or about 340 units of a neurotoxin, or about 350 units of a neurotoxin, or about 360 units of a neurotoxin, or about 370 units of a neurotoxin, or about 380 units of a neurotoxin, or about 390 units of a neurotoxin, or about 400 units of a neurotoxin, or about 410 units of a neurotoxin, or about 420 units of a neurotoxin, or about 430 units of a neurotoxin, or about 440 units of a neurotoxin, or about 450 units of a neurotoxin, or about 460 units of a neurotoxin, or about 470 units of a neurotoxin, or about 480 units of a neurotoxin, or about 490 units of a neurotoxin, or about 500 units of a neurotoxin, or the like.

In embodiments, administration can comprise a dose of not less than 10 units of a neurotoxin, not less than 20 units of a neurotoxin, not less than 30 units of a neurotoxin, not less than 40 units of a neurotoxin, not less than 50 units of a neurotoxin, not less than 60 units of a neurotoxin, not less than 70 units of a neurotoxin, not less than 80 units of a neurotoxin, not less than 90 units of a neurotoxin, not less than 100 units of a neurotoxin, not less than 110 units of a neurotoxin, not less than 120 units of a neurotoxin, not less than 130 units of a neurotoxin, not less than 140 units of a neurotoxin, not less than 150 units of a neurotoxin, not less than 160 units of a neurotoxin, or about 170 units of a neurotoxin, or about 180 units of a neurotoxin, not less than 190 units of a neurotoxin, not less than 200 units of a neurotoxin, not less than 210 units of a neurotoxin, not less than 220 units of a neurotoxin, not less than 230 units of a neurotoxin, not less than 240 units of a neurotoxin, not less than 250 units of a neurotoxin, not less than 260 units of a neurotoxin, not less than 270 units of a neurotoxin, not less than 280 units of a neurotoxin, not less than 290 units of a neurotoxin, or about 290 units of a neurotoxin, not less than 300 units of a neurotoxin, not less than 310 units of a neurotoxin, not less than 320 units of a neurotoxin, not less than 330 units of a neurotoxin, not less than 340 units of a neurotoxin, not less than 350 units of a neurotoxin, not less than 360 units of a neurotoxin, not less than 370 units of a neurotoxin, not less than 380 units of a neurotoxin, not less than 390 units of a neurotoxin, not less than 400 units of a neurotoxin, not less than 410 units of a neurotoxin, not less than 420 units of a neurotoxin, not less than 430 units of a neurotoxin, not less than 440 units of a neurotoxin, not less than 450 units of a neurotoxin, not less than 460 units of a neurotoxin, not less than 470 units of a neurotoxin, not less than 480 units of a neurotoxin, not less than 490 units of a neurotoxin, not less than 500 units of a neurotoxin, or the like.

In embodiments, administration can comprise a dose of not more than 10 units of a neurotoxin, not more than 20 units of a neurotoxin, not more than 30 units of a neurotoxin, not more than 40 units of a neurotoxin, not more than 50 units of a neurotoxin, not more than 60 units of a neurotoxin, not more than 70 units of a neurotoxin, not more than 80 units of a neurotoxin, not more than 90 units of a neurotoxin, not more than 100 units of a neurotoxin, not more than 110 units of a neurotoxin, not more than 120 units of a neurotoxin, not more than 130 units of a neurotoxin, not more than 140 units of a neurotoxin, not more than 150 units of a neurotoxin, not more than 160 units of a neurotoxin, not more 170 units of a neurotoxin, not more than 180 units of a neurotoxin, not more than 190 units of a neurotoxin, not more than 200 units of a neurotoxin, not more than 210 units of a neurotoxin, not more than 220 units of a neurotoxin, not more than 230 units of a neurotoxin, not more than 240 units of a neurotoxin, not more than 250 units of a neurotoxin, not more than 260 units of a neurotoxin, not more than 270 units of a neurotoxin, not more than 280 units of a neurotoxin, not more than 290 units of a neurotoxin, not more than 300 units of a neurotoxin, not more than 310 units of a neurotoxin, not more than 320 units of a neurotoxin, not more than 330 units of a neurotoxin, not more than 340 units of a neurotoxin, not more than 350 units of a neurotoxin, not more than 360 units of a neurotoxin, not more than 370 units of a neurotoxin, not more than 380 units of a neurotoxin, not more than 390 units of a neurotoxin, not more than 400 units of a neurotoxin, not more than 410 units of a neurotoxin, not more than 420 units of a neurotoxin, not more than 430 units of a neurotoxin, not more than 440 units of a neurotoxin, not more than 450 units of a neurotoxin, not more than 460 units of a neurotoxin, not more than 470 units of a neurotoxin, not more than 480 units of a neurotoxin, not more than 490 units of a neurotoxin, not more than 500 units of a neurotoxin, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 1 1 ng, about 2 ng and 10 ng, about 5 ng and 7 ng, and the like, into a target tissue such as a muscle.

In embodiments, neurotoxin administration can comprise a total dose of between 5 and 7 ng, between 7 and 9 ng, between 9 and 11 ng, between 11 and 13 ng, between 13 and 15 ng, between 15 and 17 ng, between 17 and 19 ng, or the like.

In embodiments, administration can comprise a total dose of not more than 5 ng, not more than 6 ng, not more than 7 ng, not more than 8 ng, not more than 9 ng, not more than 10 ng, not more than 11 ng, not more than 12 ng, not more than 13 ng, not more than 14 ng, not more than 15 ng, not more than 16 ng, not more than 17 ng, not more than 18 ng, not more than 19 ng, not more than 20 ng, or the like.

In embodiments, neurotoxin administration can comprise a total dose of not less than 5 ng, not less than 6 ng, not less than 7 ng, not less than 8 ng, not less than 9 ng, not less than 10 ng, not less than 11 ng, not less than 12 ng, not less than 13 ng, not less than 14 ng, not less than 15 ng, not less than 16 ng, not less than 17 ng, not less than 18 ng, not less than 19 ng, not less than 20 ng, or the like.

In embodiments, administration can comprise a total dose of about 0.1 ng of a neurotoxin, 0.2 ng of a neurotoxin, 0.3 ng of a neurotoxin, 0.4 ng of a neurotoxin, 0.5 ng of a neurotoxin, 0.6 n of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 ng of a neurotoxin, 3.4 ng of a neurotoxin, 3.5 ng of a neurotoxin, 3.6 n of a neurotoxin, 3.7 n of a neurotoxin, 3.8 ng of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, 11 ng of a neurotoxin, 12 ng of a neurotoxin, 13 ng of a neurotoxin, 14 ng of a neurotoxin, 15 ng of a neurotoxin, 16 ng of a neurotoxin, 17 ng of a neurotoxin, 18 ng of a neurotoxin, 19 ng of a neurotoxin, 20 ng of a neurotoxin, or the like.

In embodiments, administration can comprise a dose per administration of about 0.1 ng of a neurotoxin, 0.2 ng of a neurotoxin, 0.3 ng of a neurotoxin, 0.4 ng of a neurotoxin, 0.5 ng of a neurotoxin, 0.6 n of a neurotoxin, 0.7 ng of a neurotoxin, 0.8 ng of a neurotoxin, 0.9 ng of a neurotoxin, 1.0 ng of a neurotoxin, 1.1 ng of a neurotoxin, 1.2 ng of a neurotoxin, 1.3 ng of a neurotoxin, 1.4 ng of a neurotoxin, 1.5 ng of a neurotoxin, 1.6 ng of a neurotoxin, 1.7 ng of a neurotoxin, 1.8 ng of a neurotoxin, 1.9 ng of a neurotoxin, 2.0 ng of a neurotoxin, 2.1 ng of a neurotoxin, 2.2 ng of a neurotoxin, 2.3 ng of a neurotoxin, 2.4 ng of a neurotoxin, 2.5 ng of a neurotoxin, 2.6 ng of a neurotoxin, 2.7 ng of a neurotoxin, 2.8 ng of a neurotoxin, 2.9 ng of a neurotoxin, 3.0 ng of a neurotoxin, 3.1 ng of a neurotoxin, 3.2 ng of a neurotoxin, 3.3 ng of a neurotoxin, 3.4 ng of a neurotoxin, 3.5 ng of a neurotoxin, 3.6 n of a neurotoxin, 3.7 n of a neurotoxin, 3.8 n of a neurotoxin, 3.9 ng of a neurotoxin, 4.0 ng of a neurotoxin, 4.1 ng of a neurotoxin, 4.2 ng of a neurotoxin, 4.3 ng of a neurotoxin, 4.4 ng of a neurotoxin, 4.5 ng of a neurotoxin, 5 ng of a neurotoxin, 6 ng of a neurotoxin, 7 ng of a neurotoxin, 8 ng of a neurotoxin, 9 ng of a neurotoxin, 10 ng of a neurotoxin, or the like.

Ultimately, however, both the quantity of toxin administered and the frequency of its administration will be at the discretion of the physician responsible for the treatment and will be commensurate with questions of safety and the effects produced by the toxin.

Embodiments comprise injection of a volume of dermal filler, for example hyaluronic acid. In embodiments, the volume of hyaluronic acid composition comprises, for example, 0.3 ml, 0.4 ml, 0.5 ml, 0.6 ml, 0.7 ml, 0.8 ml, 0.9 ml, 1 ml, 1.5 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, 12 ml, 14 ml, 16 ml, 18 ml, 20 ml, 22 ml, 24 ml, 26 ml, 27 ml, 28 ml, 29 ml, 30 ml, or the like.

In embodiments, the volume of dermal filler composition comprises, for example, not more than 0.3 ml, not more than 0.4 ml, not more than 0.5 ml, not more than 0.6 ml, not more than 0.7 ml, not more than 0.8 ml, not more than 0.9 ml, not more than 1 ml, not more than 1.5 ml, not more than 2 ml, not more than 2.5 ml, not more than 3 ml, not more than 3.5 ml, not more than 4 ml, not more than 4.5 ml, not more than 5 ml, not more than 5.5 ml, not more than 6 ml, not more than 6.5 ml, not more than 7 ml, not more than 7.5 ml, not more than 8 ml, not more than 8.5 ml, not more than 9 ml, not more than 9.5 ml, not more than 10 ml, not more than 12 ml, not more than 14 ml, not more than 16 ml, not more than 18 ml, not more than 20 ml, not more than 22 ml, not more than 24 ml, not more than 26 ml, not more than 27 ml, not more than 28 ml, not more than 29 ml, not more than 30 ml, or the like.

In embodiments, the volume of dermal filler composition comprises, for example, not less than 0.3 ml, not less than 0.4 ml, not less than 0.5 ml, not less than 0.6 ml, not less than 0.7 ml, not more than 0.8 ml, not more than 0.9 ml, not less than 1 ml, not less than 1.5 ml, not less than 2 ml, not less than 2.5 ml, not less than 3 ml, not less than 3.5 ml, not less than 4 ml, not less than 4.5 ml, not less than 5 ml, not less than 5.5 ml, not less than 6 ml, not less than 6.5 ml, not less than 7 ml, not less than 7.5 ml, not more than 8 ml, not less than 8.5 ml, not less than 9 ml, not less than 9.5 ml, not more than 10 ml, not less than 12 ml, not less than 14 ml, not less than 16 ml, not less than 18 ml, not less than 20 ml, not less than 22 ml, not less than 24 ml, not less than 26 ml, not less than 27 ml, not less than 28 ml, not less than 29 ml, not less than 30 ml, or the like.

In embodiments, the treatment device or devices applying the electric charge, field, or current is applied surrounding or to the treatment area for, for example, 30 seconds, 45 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, or more, or the like.

In embodiments, the treatment device or devices applying the electric charge, field, or current is applied surrounding or to the treatment area for, for example, not less than 30 seconds, not less than 45 seconds, not less than not less than 60 seconds, not less than 1 minute, not less than 2 minutes, not less than 3 minutes, not less than 4 minutes, not less than 5 minutes, not less than 6 minutes, not less than 7 minutes, not less than 8 minutes, not less than 9 minutes, not less than 10 minutes, not less than 11 minutes, not less than 12 minutes, not less than 13 minutes, not less than 14 minutes, not less than 15 minutes, not less than 16 minutes, not less than 17 minutes, not less than 18 minutes, not less than 19 minutes, not less than 20 minutes, not less than 21 minutes, not less than 22 minutes, not less than 23 minutes, not less than 24 minutes, not less than 25 minutes, 26 minutes, not less than 27 minutes, not less than 28 minutes, not less than 29 minutes, not less than 30 minutes, not less than 31 minutes, not less than 32 minutes, not less than 33 minutes, not less than 34 minutes, not less than 35 minutes, not less than 36 minutes, not less than not less than 37 minutes, not less than 38 minutes, not less than 39 minutes, not less than 40 minutes, not less than 41 minutes, not less than 42 minutes, not less than 43 minutes, not less than 44 minutes, not less than 45 minutes, not less than 46 minutes, not less than 47 minutes, not less than 48 minutes, not less than 49 minutes, not less than 50 minutes, not less than 51 minutes, not less than 52 minutes, not less than 53 minutes, not less than 54 minutes, not less than 55 minutes, not less than 56 minutes, not less than 57 minutes, not less than 58 minutes, not less than 59 minutes, not less than 60 minutes, not less than 61 minutes, not less than 62 minutes, not less than 63 minutes, not less than 64 minutes, not less than 65 minutes, not less than 66 minutes, not less than 67 minutes, not less than 68 minutes, not less than 69 minutes, not less than 70 minutes, not less than 71 minutes, not less than 72 minutes, not less than 73 minutes, not less than 74 minutes, not less than 75 minutes, not less than 76 minutes, not less than 77 minutes, not less than 78 minutes, not less than 79 minutes, not less than 80 minutes, not less than 81 minutes, not less than 82 minutes, not less than 83 minutes, not less than 84 minutes, not less than 85 minutes, not less than 86 minutes, not less than 87 minutes, not less than 88 minutes, not less than 89 minutes, not less than 90 minutes, not less than 91 minutes, not less than 92 minutes, not less than 93 minutes, not less than 94 minutes, not less than 95 minutes, not less than 96 minutes, not less than 97 minutes, not less than 98 minutes, not less than 99 minutes, not less than 100 minutes, not less than 101 minutes, not less than 102 minutes, not less than 103 minutes, not less than 104 minutes, not less than 105 minutes, 106 minutes, 1 not less than 07 minutes, not less than 108 minutes, not less than 109 minutes, not less than 110 minutes, not less than 111 minutes, not less than 112 minutes, not less than 113 minutes, not less than 114 minutes, not less than 115 minutes, not less than 116 minutes, not less than 117 minutes, not less than 118 minutes, not less than 119 minutes, not less than 120 minutes, or more, or the like.

In embodiments, the treatment device or devices applying the electric charge, field, or current is applied surrounding or to the treatment area for, for example, not more than 30 seconds, not more than 45 seconds, not more than not more than 60 seconds, not more than 1 minute, not more than 2 minutes, not more than 3 minutes, not more than 4 minutes, not more than 5 minutes, not more than 6 minutes, not more than 7 minutes, not more than 8 minutes, not more than 9 minutes, not more than 10 minutes, not more than 11 minutes, not more than 12 minutes, not more than 13 minutes, not more than 14 minutes, not more than 15 minutes, not more than 16 minutes, not more than 17 minutes, not more than 18 minutes, not more than 19 minutes, not more than 20 minutes, not more than 21 minutes, not more than 22 minutes, not more than 23 minutes, not more than 24 minutes, not more than 25 minutes, not more than 26 minutes, not more than 27 minutes, not more than 28 minutes, not more than 29 minutes, not more than 30 minutes, not more than 31 minutes, not more than 32 minutes, not more than 33 minutes, not more than 34 minutes, not more than 35 minutes, not more than 36 minutes, not more than 37 minutes, not more than 38 minutes, not more than 39 minutes, not more than 40 minutes, not more than 41 minutes, not more than 42 minutes, not more than 43 minutes, not more than 44 minutes, not more than 45 minutes, not more than 46 minutes, not more than 47 minutes, not more than 48 minutes, not more than 49 minutes, not more than 50 minutes, not more than 51 minutes, not more than 52 minutes, not more than 53 minutes, not more than 54 minutes, not more than 55 minutes, not more than 56 minutes, not more than 57 minutes, not more than 58 minutes, not more than 59 minutes, not more than 60 minutes, not more than 61 minutes, not more than 62 minutes, not more than 63 minutes, not more than 64 minutes, not more than 65 minutes, not more than 66 minutes, not more than 67 minutes, not more than 68 minutes, not more than 69 minutes, not more than 70 minutes, not more than 71 minutes, not more than 72 minutes, not more than 73 minutes, not more than 74 minutes, not more than 75 minutes, not more than 76 minutes, not more than 77 minutes, not more than 78 minutes, not more than 79 minutes, not more than 80 minutes, not more than 81 minutes, not more than 82 minutes, not more than 83 minutes, not more than 84 minutes, not more than 85 minutes, not more than 86 minutes, not more than 87 minutes, not more than 88 minutes, not more than 89 minutes, not more than 90 minutes, not more than 91 minutes, not more than 92 minutes, not more than 93 minutes, not more than 94 minutes, not more than 95 minutes, not more than 96 minutes, not more than 97 minutes, not more than 98 minutes, not more than 99 minutes, not more than 100 minutes, not more than 101 minutes, not more than 102 minutes, not more than 103 minutes, not more than 104 minutes, not more than 105 minutes, not more than 106 minutes, not more than 107 minutes, not more than 108 minutes, not more than 109 minutes, not more than 110 minutes, not more than 111 minutes, not more than 112 minutes, not more than 113 minutes, not more than 114 minutes, not more than 115 minutes, not more than 116 minutes, not more than 117 minutes, not more than 118 minutes, not more than 119 minutes, not more than 120 minutes, or more, or the like.

Similarly, dermal fillers can be localized via the disclosed methods. For example, in embodiments, at physiological pH values, hyaluronic acid carries a net negative charge. In embodiments, by applying a positive source charge to a location, the dermal filler can be “fixed” in that location. Similarly, by applying a negative source charge, for example around part or all of the perimeter of a treatment site, the dermal filler can be excluded or repelled from a location. Dermal fillers suitable for use with disclosed methods and systems include, for example, those containing Hyaluronic Acid (HA), Calcium Hydroxyapatite (CaHA), Poly-L-lactic Acid, Polymethylmethacrylate (PMMA), Autologous fat injections (facial fat grafting), and combinations thereof.

Disclosed embodiments can be used to limit the amount of non-therapeutic materials to be injected into a patient. For example, in embodiments, the vessel containing the material to be injected is subjected to an electromagnetic field that attracts the therapeutic material, and the injection material is withdrawn from the vessel in the vicinity of the attractive electromagnetic field.

Disclosed embodiments comprise methods of controlling release from “depot” drug administration. For example, “depot” drug administration refers to an injection formulation of a medication which releases slowly over time to permit less frequent administration of a medication. They are designed to increase medication adherence and consistency, especially in patients who commonly forget to take their medicine. Depot injections are available for many types of drugs, including antipsychotics and hormones. By controlling dissipation of a depot injection via the use of disclosed attractive and repellent forces, the time-release curve of the drug can be controlled.

### Administered Active Agents

As should now be evident, administration of therapeutics can occur before or after applying the electrodes but is preferably performed before applying electric charge to the electrodes. In embodiments, the administered, for example injected materials, can comprise pharmaceutical compositions, which include an active agent such as a charged active agent. For example, suitable pharmaceutical compositions can comprise any materials typically administered via injection (to include needle-less injection). Such compositions can comprise, for example, biologics such as neurotoxins or components thereof, anesthetics, analgesics, dermal fillers, and the like.

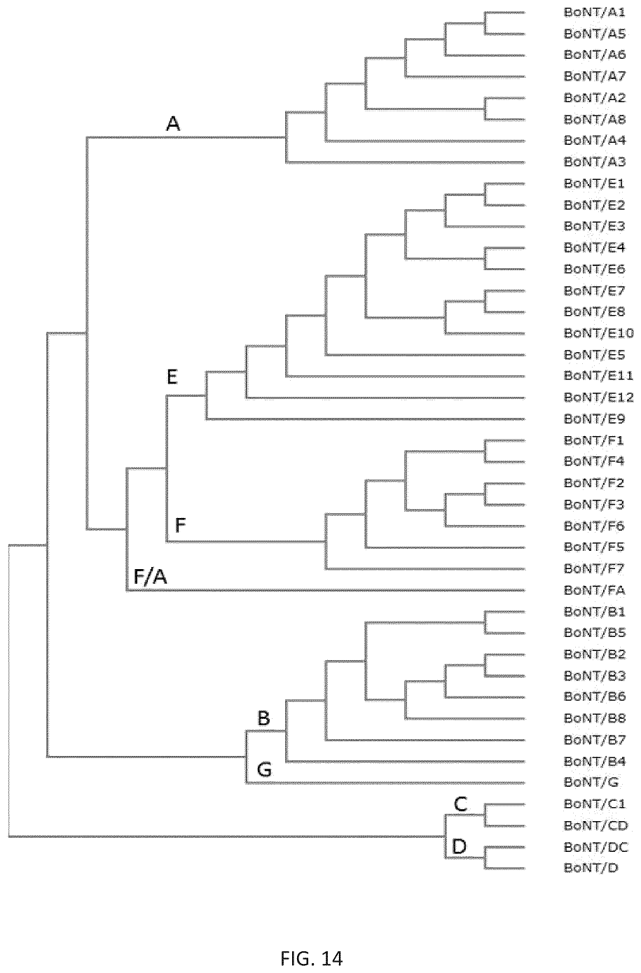

Botulinum neurotoxins suitable for use with disclosed methods and systems include, for BoNT/A, BoNT/B, BONT/C, BONT/D, BONT/E, BONT/F, BONT/G, BONT/H, and combinations thereof, both as the “complex” such as the 900 kd BoNT/A complex or the 150 kd neurotoxin component of the 900 kd BoNT/A complex. For example, FIG. 14 groups together exemplary botulinum toxins suitable for use in disclosed methods and compositions:

Further suitable neurotoxins can comprise the “light chain” of a botulinum toxin. Embodiments can comprise a combination of neurotoxins, for example BoNT/A and BoNT/E. Embodiments can comprise the dissociation of the heavy chain and the light chain, either prior to or following administration. Disclosed embodiments can comprise use of an electric charge, field, or current to dissociate a neurotoxin from accessory proteins or formulation components, for example HSA, either prior to or following an administration, for example an injection. Embodiments comprise aligning the dipole of a botulinum toxin to provide the correct orientation for binding.

In embodiments, when the neurotoxin complex is injected, the complex dissociates under physiological pH to release the neurotoxin component. In such cases the strength and orientation of the attractive and repellent forces can be determined based on the properties (such as pl) of the neurotoxin component itself.

Dermal fillers suitable for use with disclosed methods and systems include, for example, those containing Hyaluronic Acid (HA), Calcium Hydroxylapatite (CaHA), Poly-L-lactic Acid, Polymethylmethacrylate (PMMA), Autologous fat injections (facial fat grafting), and combinations thereof.

Embodiments can also comprise adjusting the isoelectric point (pl) of an injected material, for example using recombinant technologies. For example, disclosed embodiments comprise an engineered clostridial toxin comprising at least one amino acid modification, wherein said at least one amino acid modification increases the isoelectric point (pl) of the engineered clostridial toxin to a value that is at least 0.2 (for example, at least 0.2, 0.3, 0.4, 0.5, 0.6, 0.7, 0.8, 0.9 or 1) pl units higher than the pl of an otherwise identical clostridial toxin lacking said at least one amino acid modification. In one embodiment, said at least one amino acid modification increases the pl of the engineered clostridial toxin to a value that is at least 0.4 pl units higher than the pl of an otherwise identical clostridial toxin lacking said at least one amino acid modification. In one embodiment, said at least one amino acid modification increases the pl of the engineered clostridial toxin to a value that is at least 0.5 pl units higher than the pl of an otherwise identical clostridial toxin lacking said at least one amino acid modification. In one embodiment, said at least one amino acid modification increases the pl of the engineered clostridial toxin to a value that is at least 0.6 pl units higher than the pl of an otherwise identical clostridial toxin lacking said at least one amino acid modification. In one embodiment, said at least one amino acid modification increases the pl of the engineered clostridial toxin to a value that is at least 0.8 pl units higher than the pl of an otherwise identical clostridial toxin lacking said at least one amino acid modification. In one embodiment, said at least one amino acid modification increases the pl of the engineered clostridial toxin to a value that is at least 1 pl unit higher than the pl of an otherwise identical clostridial toxin lacking said at least one amino acid modification.

Further disclosed embodiments comprise an engineered clostridial toxin comprising at least one amino acid modification, wherein said at least one amino acid modification decreases the pl of the engineered clostridial toxin to a value that is at least 0.2 (for example, at least 0.2, 0.3, 0.4, 0.5, 0.6, 0.7, 0.8, 0.9 or 1) pl units lower than the pl of an otherwise identical clostridial toxin lacking said at least one amino acid modification. In one embodiment, said at least one amino acid modification decreases the pl of the engineered clostridial toxin to a value that is at least 0.4 pl units lower than the pl of an otherwise identical clostridial toxin lacking said at least one amino acid modification. In one embodiment, said at least one amino acid modification decreases the pl of the engineered clostridial toxin to a value that is at least 0.5 pl units lower than the pl of an otherwise identical clostridial toxin lacking said at least one amino acid modification. In one embodiment, said at least one amino acid modification decreases the pl of the engineered clostridial toxin to a value that is at least 0.6 pl units lower than the pl of an otherwise identical clostridial toxin lacking said at least one amino acid modification. In one embodiment, said at least one amino acid modification decreases the pl of the engineered clostridial toxin to a value that is at least 0.8 pl units lower than the pl of an otherwise identical clostridial toxin lacking said at least one amino acid modification. In one embodiment, said at least one amino acid modification decreases the pl of the engineered clostridial toxin to a value that is at least 1 pl unit lower than the pl of an otherwise identical clostridial toxin lacking said at least one amino acid modification.

Though primarily discussed with respect to injection, charged active agents can be administered using iontophoresis, iontophoresis is a process of transdermal drug delivery by use of a voltage gradient on the skin. During iontophoresis, a small electric current is applied to an iontophoretic chamber placed on the skin, containing a charged active agent and its solvent vehicle. Another chamber (or a skin electrode) carries the return current. The positively charged chamber, called the anode, will repel a positively charged chemical species, whereas the negatively charged chamber, called the cathode, will repel a negatively charged species into the skin.

Iontophoresis techniques can safely “drive” molecules over 1 cm into tissue, for example beneath the skin, using safe, well-tolerated voltages and currents. Such depths are suitable for methods disclosed herein, as many administered, for example injected, materials are administered just below the skin or at a minimum depth intramuscularly. Therefore, these safe, well-tolerated voltages and currents used in iontophoresis can also be used to control material dissipation by, rather than using a repellent force to drive a material into the skin, using that repellent force to “fence”, drive, or limit the material to a desired treatment area. In addition to the repellent force, an attractive force can also be used to localize an administered material, as well as providing a “return” for any currents produced.

Further embodiments comprise iontophoresis patches that “phase switch.” For example, the iontophoresis patch is employed with a disclosed administered material, with the patch employing an electrode comprising a repellent charge to drive the material into the skin, and a return electrode forming at least part of the perimeter of the device. After the material is substantially or completely driven into the skin, the polarity of the device reverses, such that the repellent electrode then forms the perimeter, thus containing the area of dissipation of the material.

### EXAMPLES

### Example 1

### Starch Test Quantification

8 facial neurotoxin injections are administered to subjects to test for a starch test signal following intramuscular injection. Each of the 8 injections shows a signal on the starch test, confirming that even with an intramuscular injection, the injected material affects non-muscular tissue.

### Example 2

### Use of an Electric Field to Minimize Neurotoxin Spread

As seen in FIGS. 9 and 10, the device of FIG. 9 (comprising an attractive and a repellent electrode placed opposite each other at either end of the long axis) is placed on the skin on one side of the face of each of two subjects, providing a test side (with electrodes) and control side (without electrodes). 20 U of BoNT/A is injected in the gap region between the two electrodes.

FIG. 10 shows the results of the injections as quantified with a starch test performed two days after injection. The white areas show where the toxin has limited perspiration, the dark areas indicate perspiration. Subject 1 (left); low power electrodes (creating minimal muscle contractions) were used on right side of the face (left side of image). The test injection shows better spread as well as migrating “up” toward the attractive electrode as compared to the negative control (no electrodes) injected in the same horizontal plane.

Subject 2 (right); medium power electrodes used on left side of the face (right side of image. The test injection shows better spread as well as migrating “down” toward the attractive electrode as compared to the negative control (no electrodes) injected in the same horizontal plane (the electrodes were reversed on the two subjects).

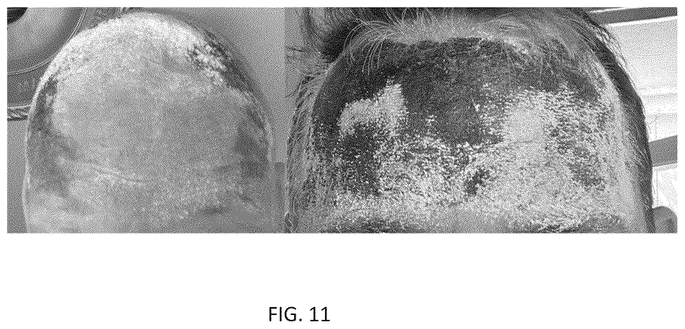

FIG. 11 shows results follow up starch test results from Subject1 (left) and Subject 2 (right) approximately 20 weeks after the study conducted with the electrode configuration of FIG. 9 confirming the localized toxin remained in its targeted position.

### Example 3

### Use of an Electric Field to Minimize Neurotoxin Spread

A 44 year old male patient is going to be treated to minimize glabellar lines with BoNT/A. Prior to administering the toxin via needle injection, the doctor applies a disclosed embodiment that generates a repellent electric charge between the intended injection sites and the patient's eyes. An attractive charge is established toward the patient's hairline. An electric field is established between the two charges.

Then, the doctor injects 5 sites with 4 U of the BoNT/A to the typical glabellar lines treatment sites. The patient wears the embodiment producing the electric field for 30 minutes. The patient experiences no ptosis.

### Example 4

### Use of an Electric Field to Minimize Neurotoxin Spread

A 54 year old female patient is going to be treated to minimize glabellar lines with BONT/A. Prior to administering the toxin via needle injection, the doctor applies a disclosed embodiment that generates a repellent electric charge between the intended injection sites and the patient's eyes. An attractive charge is established toward the patient's hairline. An electric field is established between the two charges.

Then, the doctor injects 5 sites with 4 U of the BoNT/A to the typical glabellar lines treatment sites. The patient wears the embodiment producing the electric field for 20 minutes. The patient experiences no ptosis.

### Example 5

### Use of an Electric Field to Direct Neurotoxin Spread

A 32 year old female patient is going to be treated to minimize glabellar lines with BoNT/B. Prior to administering the toxin via needle injection, the doctor applies a disclosed embodiment that generates a repellent electric charge between the eyes and the intended injection sites. An attractive electric charge is applied at the hairline. An electric field is established between the two charges.

Then, the doctor injects 5 sites with 4 U of the BoNT/B to the typical glabellar lines treatment sites. The patient wears the embodiment producing the electric field for 45 minutes. The patient experiences no ptosis.

### Example 6

### Use of an Electric Field to Direct Neurotoxin Spread

A 27 year old female patient is going to be treated to minimize crows feet with BoNT/A. Prior to administering the toxin via needle injection, the doctor applies a disclosed embodiment comprising a platinum electrode to produce a repellent electric charge between the intended injection sites and the eyes. An attractive electric charge is applied between the injection sites and the ears. An electric field is established between the two charges.

Then, the doctor injects 7 sites with 3 U of the BoNT/A to the typical glabellar lines treatment sites. The patient wears the embodiment producing the electric field for 45 minutes. The patient experiences no ptosis.

### Example 7

### Use of an Electric Field to Minimize Neurotoxin Spread

A 47 year old male patient is going to be treated for migraine. Prior to administering the toxin via needle injection to the trapezius muscle, the doctor applies a disclosed embodiment comprising a repellent electric charge to prevent neurotoxin dissipation toward the midline.

An attractive electric charge is applied further down the trapezius toward the shoulder. An electric field is established between the two charges. The applied voltage level causes minimal muscle contraction.

Then, the doctor injects 5 sites between the charges with 4 U of the BoNT/A to the trapezius. The patient wears the embodiment producing the positive electric field for one hour. The patient experiences no neurotoxin dissipation toward the midline, and the effect onset of the neurotoxin is accelerated by the applied electric field.

### Example 8

### Use of an Electric Field to Minimize Neurotoxin Spread

A 33 year old female patient is going to be treated to minimize crows feet with BoNT/A. Prior to administering the toxin via needle injection, the doctor applies a disclosed embodiment comprising a zinc electrode that generates a positive electric field in a perimeter between the intended injection sites and the eyes. Then, the doctor injects 5 sites with 4 U of the BoNT/A to the typical crow's feet treatment sites.

Following the injections, the doctor applies a disclosed embodiment comprising a silver electrode that generates a negative electric field toward the hairline. The patient wears the embodiments producing the electric field for 30 minutes. The patient experiences no ptosis.

### Example 9

### Use of an Electric Field to Minimize Neurotoxin Spread

A 54 year old female patient is going to be treated to minimize crows feet with BONT/A. Prior to administering the toxin via needle injection, the doctor applies a disclosed embodiment comprising a electrode that generates a positive electric charge in a perimeter surrounding the intended injection sites. Then, the doctor injects 5 sites with 5 U of the BoNT/E to the typical crows feet treatment sites. Following the injections, the doctor applies a disclosed embodiment comprising an electrode that generates a negative electric field directly on top of the injection sites. The patient wears the embodiments producing the positive and negative charges for 30 minutes. The patient experiences no ptosis.

### Example 10

### Use of an Electric Field to Minimize Neurotoxin Spread

A 33 year old female patient is going to be treated to minimize neck lines with BoNT/B. Prior to administering the toxin via needle injection, the doctor applies a disclosed embodiment that generates a repellent electric charge between the intended injection sites and the jaw. An attractive charge is placed lower on the neck to increase dissipation away from the jaw muscles. An electric field is established between the two charges.

Then, the doctor injects 5 sites with 4 U of the BoNT/B to the treatment sites. The patient wears the embodiments producing the electric field for 30 minutes. The patient experiences no spread of the toxin from the treatment site.

### Example 11

### Use of an Electric Field to Minimize Dermal Filler Spread

A 54 year old male patient is going to be treated with hyaluronic acid. Prior to administering the filler via needle injection, the doctor applies a disclosed embodiment that generates a repellent electric charge in a perimeter surrounding the intended injection sites. The patient wears the embodiment producing the electric field for an hour. The patient experiences no diffusion of the filler.

### Example 12

### Use of an Electric Field to Minimize Dermal Filler Spread

A 43 year old female patient is going to be treated with hyaluronic acid. Prior to administering the filler via needle injection, the doctor applies a disclosed embodiment that generates a repellent electric field in a perimeter surrounding the intended injection sites. Following the injections, the doctor applies a disclosed embodiment that generates an attractive electric field directly on top of the injection sites. The patient wears the embodiments producing the attractive and repellent electric fields for 30 minutes. The patient experiences no diffusion of the filler.

### Example 13

### Use of an Ems to Minimize Neurotoxin Spread

A 33 year old female patient is going to be treated to minimize crows feet with BoNT/A. Prior to administering the toxin via needle injection, the doctor applies a EMS device to minimize spread of the neurotoxin. Prior to injection, positive electrodes of the EMS are applied to repel the toxin from beyond the treatment area, while the negative electrode is placed toward the ears. The intensity of the EMS is increased until the patient reports a tingling sensation.

### Example 14

### Use of a Ems to Minimize Neurotoxin Spread

10 units of cold BoNT/A is administered to a 48 year old male in the upper back region. The injection is made between (1″ spaced) attractive and repellent electrodes and a low muscle-contraction level of voltage is applied. Composition dissipation is monitored through the use of a FLIR camera, which shows the composition dissipating away from the repellent electrode and toward the attractive electrode. A starch test is performed three weeks post-injection, which confirms the FLIR results.

### Example 15

### Use of Ems to Minimize Neurotoxin Spread

10 units of cold BoNT/A was administered to a 52 year old male in the forehead region. The patient wore a linear strip repellent electrode above the eyes, and a linear strip attractive electrode just below the hairline (FIG. 7). Two injections were made between the electrodes, spaced linearly ½″ apart, and a non-muscle-contraction level of voltage was applied. Composition dissipation was monitored through the use of a FLIR camera, which showed the composition dissipating away from the repellent electrode and toward the attractive electrode. A starch test was performed three weeks post-injection showing dissipation away from the repellent electrode and towards the attractive electrode (FIG. 8). Lateral spreading along the attractive electrode was also observed (FIG. 8).

### Example 16

### Use of Ems to Minimize Neurotoxin Spread

20 units of cold BoNT/A was administered to a 52 year old male in the forehead region. 10 units was injected on each side of the face, with the right side of the face utilizing the electrode system of FIG. 9. The “upper” electrode provided the attractive charge while the “lower” electrode provided the repellent charge to prevent dissipation toward the eye. The injections were made 1 cm apart spaced vertically, with the injections on the electrode side were made within the diamond-shaped cutout. A starch test was performed two days post-injection showing dissipation away from the repellent electrode and towards the attractive electrode. The test injection showed better spread as well as migrating “up” toward the attractive electrode.

### Example 17

### Use of Ems to Minimize Neurotoxin Spread

20 units of cold BoNT/A was administered to a 48 year old male in the forehead region. 10 units was injected on each side of the face, with the right side of the face utilizing the electrode system of FIG. 9. The “upper” electrode provided the attractive charge while the “lower” electrode provided the repellent charge to prevent dissipation toward the eye. The injections were made 1 cm apart spaced vertically, with the injections on the electrode side were made within the diamond-shaped cutout. A starch test was performed two days post-injection showing dissipation away from the repellent electrode and towards the attractive electrode. The test injection showed better spread as well as migrating “down” toward the attractive electrode as compared to the negative control.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, including the best mode known to the inventor for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

What is claimed is:

1) A device for directing dissipation of a charged active agent after injection, the device comprising two electrodes spaced apart from one another on a flexible substrate so that a directional electric field can be generated between the two electrodes, the flexible substrate configured for placement against the skin of a subject and comprising:
a through gap sized for permitting passage of an injection device, and optionally positioned between the two electrodes; or
a tearaway portion configured to tear away to form a through gap sized for permitting passage of an injection device, and optionally positioned between the two electrodes.

2) The device of claim 1, wherein the flexible substrate is shaped for placement above the eyes of the subject and so that the through gap is positioned to permit intramuscular injection of a charged composition at the subject's forehead.

3) The device of claim 1, wherein the tearaway portion is a tearaway perforation that extends between the two electrodes.

4) The device of claim 1, wherein the flexible substrate comprises a tether that tethers the two electrodes together.

5) The device of claim 1, wherein a first of the two electrodes is encircled by a second of the two electrodes.

6) The device of claim 1, wherein the two electrodes are substantially parallel to one another.

7) The device of claim 1, further comprising a power supply that supplies opposing charges to the two electrodes to power the directional electric field.

8) The device of claim 7, wherein the power supply is battery powered and attached to a same flexible substrate as the two electrodes.

9) The device of claim 7, wherein the power supply is configured for mounting to the subject's face or head.

10) The device of claim 1, comprising electrode connectors that differ in shape between the two electrodes.

11) A device for directing dissipation of a charged active agent after injection, the device comprising two electrodes spaced apart from one another on a perforated substrate that is configured to tear away to separate the two electrodes from one another, thereby forming a through gap sized to permit passage of an injection device, the substrate comprising an adhesive backing configured for positioning the two electrodes against the skin of a subject for the generation of a directional electric field.

12) A method of directing dissipation of a charged active agent in a subject, the method comprising: applying the device of claim 1 against the skin of the subject, injecting a charged active agent through the through gap, and applying opposing charges to the two electrodes, thereby creating a directional electric field between the two electrodes and approximately parallel to the skin so that the polarity encourages dissipation towards one of the two electrodes and away from another.

13) The method of claim 12, wherein the charged active agent comprises a neurotoxin, optionally a botulinum toxin (BoNT) selected from the group consisting of BoNT/A, BoNT/E, and BoNT/B.

14) The method of claim 12, wherein the opposing charges are pulsed.

15) The method of claim 12, wherein the device is positioned at a muscle selected from the group consisting of the procerus muscle, the corrugator muscle, the frontalis muscle, the occipitalis muscle, and the trapezius muscle.

16) The method of claim 12, wherein the device is positioned on the subject's face.

17) A method of directing dissipation of a charged active agent in a subject, the method comprising: providing the device of claim 11, tearing the perforation to separate the two electrodes from one another, applying the device against the skin of the subject, injecting a charged active agent through the through gap, and applying opposing charges to the two electrodes, thereby creating a directional electric field between the two electrodes and approximately parallel to the skin so that the polarity encourages dissipation towards one of the two electrodes and away from another.

18) A method for directing dissipation of a charged active agent, the method comprising:
a) placing two electrodes against a subject's skin, the two electrodes configured to receive opposing charges to generate a directional electric field between the two electrodes and approximately parallel to the skin;
b) subcutaneously or intramuscularly administering the charged active agent between the two electrodes, the charged active agent comprising a net negative or net positive charge at physiological pH; and
c) applying a positive charge to one of the two electrodes and a negative charge to another of the two electrodes to produce the directional electric field, thereby electrically directing dissipation of the administered charged active agent towards one of the two electrodes and away from another of the two electrodes.

19) The method of claim 18, wherein the two electrodes are placed 1-5 cm apart from one another.

20) A method for directing dissipation of a charged active agent, the method comprising:
a) subcutaneously or intramuscularly administering a charged active agent into a subject, the charged active agent comprising a net negative or net positive charge at physiological pH;
b) placing two electrodes against the subject's skin, the two electrodes configured to receive opposing charges to generate a directional electric field approximately parallel to the skin and at the administered charged active agent; and
c) applying a positive charge to one of the two electrodes and a negative charge to another of the two electrodes to produce the directional electric field, thereby electrically directing dissipation of the administered charged active agent towards one of the two electrodes and away from the oppositely charged electrode.
